(19) United States
(12) Patent Application Publication
(10) Pub. No.: US 2014/0073678 A1
(43) Pub. Date: Mar. 13, 2014

(54) ANTI-PAIN AND ANTI-NAUSEA AND/OR VOMITING COMBINATORIAL COMPOSITIONS

(71) Applicant: MONOSOL RX, LLC, Warren, NJ (US)

(72) Inventors: Eric Dadey, Furlong, PA (US); Alexander Mark Schobel, Whitehouse Station, NJ (US); Daniel R. Barber, Summit, NJ (US); Garry L. Myers, Kingsport, TN (US)

(73) Assignee: MONOSOL RX, LLC, Warren, NJ (US)

(21) Appl. No.: 13/843,718

(22) Filed: Mar. 15, 2013

Related U.S. Application Data
(60) Provisional application No. 61/700,146, filed on Sep. 12, 2012.

(51) Int. Cl. A61K 31/4196; A61K 31/4178

(52) U.S. Cl. CPC A61K 31/4196; A61K 31/4178
USPC 514/383

(57) ABSTRACT

In one aspect, the present invention discloses combinational compositions for treating users experiencing symptoms associated with a migraine or other central nervous system related pain disorder that can cause or exacerbate nausea and/or vomiting or other central nervous system related pain disorder that can cause or exacerbate nausea and/or vomiting. The combinational composition includes a first pharmaceutical active component for treating pain, and a second pharmaceutical active component for treating nausea and/or vomiting and/or vomiting in a user.

### CROSS-REFERENCE TO RELATED APPLICATION

This application claims the benefit of priority under 35 U.S.C. §119 of U.S. Provisional Application Ser. No. 61/70,146 filed on Sep. 12, 2012 the content of which is relied upon and incorporated herein by reference in its entirety.

### FIELD OF THE INVENTION

The present invention is related to combinational compositions for delivery of actives. Specifically, the combinational composition includes a first pharmaceutical active component for treating pain, and a second pharmaceutical active component for treating nausea and/or vomiting and/or vomiting in a user. The composition is desirably effective at treating users experiencing symptoms associated with a migraine or other central nervous system related pain disorder that can cause or exacerbate nausea and/or vomiting or other central nervous system related pain disorder that can cause or exacerbate nausea and/or vomiting.

### BACKGROUND

People who suffer from migraines not only experience high levels of pain, but also experience other symptoms, including nausea and/or vomiting. Some report as high as 75% of migraine patients suffer from both conditions. Treatments for such symptoms typically include administration of two separate drugs, one to treat pain and a second to treat nausea and/or vomiting. There is currently a need for a combinatorial composition that is effective at treating not only the pain associated with a migraine or other central nervous system related pain disorder but also the nausea and/or vomiting experienced by a user. A further unmet need exists for not only treating the feeling of nausea and/or vomiting but also providing a faster onset of action from the migraine medication to reduce or eliminate pain.

### SUMMARY OF THE INVENTION

In one aspect, the present invention discloses combinational compositions for delivery of actives. The combinational composition includes a first pharmaceutical active component for treating pain, and a second pharmaceutical active component for treating nausea and/or vomiting and/or vomiting in a user. The composition is desirably effective at treating users experiencing symptoms associated with a migraine or other central nervous system related pain disorder that can cause or exacerbate nausea and/or vomiting or other central nervous system related pain disorder that can cause or exacerbate nausea and/or vomiting.

In one aspect, the present invention discloses a film product including a polymer component, a therapeutically effective amount of rizatriptan or a pharmaceutically acceptable salt thereof; and a therapeutically effective amount of ondansetron or a pharmaceutically acceptable salt thereof.

In another aspect, the present invention discloses methods of treating users experiencing symptoms associated with a migraine or other central nervous system related pain disorder that can cause or exacerbate nausea and/or vomiting or other central nervous system related pain disorder that can cause or exacerbate nausea and/or vomiting, by administering therapeutically effective amounts of the instant combination.

### BRIEF DESCRIPTION OF THE DRAWINGS

The present invention is further described by FIG. 1 and FIG. 2 in the application.

FIG. 1 is a plot of plasma rizatriptan levels versus time in minutes after dosing in mini pigs and indicates the therapeutic window.

FIG. 2 is a plot of plasma rizatriptan levels versus time in minutes after dosing in subjects and indicates the rizatriptan levels in rizatriptan films and combination films.

FIG. 3 is a plot of plasma rizatriptan levels versus time in hours after dosing in mini pigs with a combination rizatriptan/ondansetron (10/8 mg) formulation.

FIG. 4 is a plot of plasma ondensetron levels versus time in hours after dosing in mini pigs with a combination rizatriptan/ondansetron (10/8 mg) formulation.

### DETAILED DESCRIPTION

As explained above, a significant number of migraine sufferers not only experience pain symptoms with a migraine but also nausea and/or vomiting. The nausea and/or vomiting experienced by an individual can be so severe that it causes sufferers to limit activities and is therefore a major cause of migraine disability. The feeling of nausea can occur by itself, or it may induce vomiting by a sufferer.

As used herein, the terms “pharmaceutical”, “medicament”, “drug” and “active” may be used interchangeably, and refer to a substance or composition useful for the prevention or treatment of a condition. The terms may include pharmaceuticals, neutraceuticals, cosmetic agents, biologic agents, bioeffective substances, and the like.

The pharmaceutical dosage form of the present invention may include a dissolvable tablet or pill, or alternatively may include a dissolvable or disintegrable film product, or may include a patch. The dosage form may include a combination of dissolvable tablet and dissolvable or disintegrable film, secured to each other, such as through lamination or chemical bonding.

It will be understood that the term “film” includes delivery systems of any thickness, including films and film strips, sheets, discs, wafers, and the like, in any shape, including rectangular, square, or other desired shape. The film may be in the form of a continuous roll of film or may be sized to a desired length and width. The films described herein may be any desired thickness and size suitable for the intended use. For example, a film of the present invention may be sized such that it may be placed into the oral cavity of the user. Other films may be sized for application to the skin of the user, i.e., a topical use. For example, some films may have a relatively thin thickness of from about 0.1 to about 10 mils, while others may have a somewhat thicker thickness of from about 10 to about 30 mils. For some films, especially those intended for topical use, the thickness may be even larger, i.e., greater than about 30 mils. In addition, the term “film” includes single-layer compositions as well as multi-layer compositions, such as laminated films, coatings on films and the like. The composition in its dried film form maintains a uniform distribution of components through the processing of the film. Films may include a pouch or region of medicament between two films.

The term “patch” as used herein is intended to include multi-layered film products, where the first layer (or “backing layer”) is a film product that has a slower rate of dissolution than the second layer (or “active layer”). Patches described herein generally include the first and second layers adhered or laminated to each other, where the second layer has a smaller length and/or width of the first layer, such that at least a portion of the surface of the first layer is visible outside of the second layer.

Products formed by the present invention may be suitable for administration to at least one region of the body of the user, such as mucosal regions or regions within the body of the user, such as on the surface of internal organs. In some embodiments of the invention, the products are intended for oral administration. In other embodiments, the films are intended for topical administration. As used herein, the term “topical agent” is meant to encompass active agents that are applied to a particular surface area. For example, in one embodiment, a topical agent is applied to an area of the skin. In other embodiments, the topical agent may also be applied to mucosal areas of the body, such as the oral (e.g., buccal, sublingual, tongue), vaginal, ocular and anal areas of the body. It may be understood that films of the present invention may be capable of being applied to more than one mucosal area of the body simultaneously, such as more than one oral mucosal surface. Examples of more than one surface can include, for example, under tongue—floor of mouth, lingual—hard pallet, and buccal—gingival. In still other embodiments, the topical agent may be applied to an internal organ or other body surface of the user, such as during surgery, where the agent may be removed or left within the body after surgery is complete. In other embodiments, a topical agent may be applied to a hard surface, such as a particular surface area in need of treatment. In other embodiments, the films of the present invention are ingestible, and are intended to be placed in the mouth of the user and swallowed as the film disintegrates and/or dissolves.

Most preferably, the present invention includes a product that may be placed in the sublingual region of the user, allowing the product to quickly release a first pharmaceutical active agent, and also allowing dissolution of the product slowly to release a second pharmaceutical active agent. As will be described in more detail below, the first pharmaceutical active agent may be desirably absorbed through the mucosal surface of the user, while the second pharmaceutical active agent may be desirably swallowed and released into the gastrointestinal region of the user.

The pharmaceutical active agents may be dispersed throughout the product, or it may be deposited onto one or more surfaces of the product. In either way, it is desirable that the amount of pharmaceutical active agent per unit area is substantially uniform throughout the product. The “unit area” is intended to include a suitable unit area, such as the area of one typical dosage unit. It is desired that the products of the present invention where the medicament is dispersed throughout the film include a uniformity of component distribution throughout the volume of a given product. Such uniformity includes a substantially uniform amount of medicament per unit volume of the product where the unit volume is one dosage form, whether the medicament is within the matrix of the product or coated, laminated, deposited or stabilized on one or more surfaces thereof.

The uniformity may include substantial uniformity of content between and among individual dosages. When such products, such as films, are cut into individual units, the amount of the agent in the unit can be known with a great deal of accuracy. For the products formed herein, it is understood by one of ordinary skill in the art that the resulting product is not required to be exactly 100% uniform. All that is required is that the products be “substantially uniform”, i.e., a slight amount of non-uniformity is understood to be acceptable. “Substantially uniform” may include, for example, a product that is about 90% uniform in content from one region of the product to another, or a product that is about 95% uniform in content from one region of the product to another, and most desirably about 99% uniform in content from one region. It is desirable that any individual product products formed by the present invention (i.e., products having a substantially similar mass and volume) be substantially uniform in content with respect to each other. That is, the individual products (including individual dosages of approximately equal sizes) formed by the present invention should have approximately the same content composition as each other product. In some embodiments, uniformity may be determined by comparing two regions of the same product having substantially the same area, for example, taking two 1 cm³ regions of a film product and comparing for uniformity. Of course, it will be understood that some deviation is to be expected during the manufacturing process, but desirably the individual products should be at least 90% uniform in content with respect to each other. In other words, “substantially uniform” may mean that individual products should vary by no more than about 10% with respect to each other. In some embodiments, “substantially uniform” may mean that individual products should vary by no more than about 5% with respect to each other.

The inventive product may be in the form of a large scale film product, such as a large roll of film from which smaller films are prepared. In such embodiments, the inventive product has a suitable and desirable level of uniformity such that the user can feel confident that no matter where unit doses are obtained from the roll of film, the resulting unit doses will have a substantially uniform active content as defined by no more than a 10% variance of the active composition as measured by substantially equally-sized individual unit doses from the film. Large, commercial-scale processes may form rolls of film from which at least 500,000 individual film dosages are cut. Each individual film unit dosage is sized approximately the same, and preferably contain the desired amount of active dosage contained therein. As can be appreciated, the need for compositional uniformity is critically important, particularly for regulated products, such as pharmaceuticals. The desired amount of active contained in each individual dosage unit is referred to as the “label claim”, and it is desired that there be no more than a 10% variance from that label claim (i.e., from 90% to 110% of the label claim).

Uniformity of medicament throughout the product is important in administering an accurate and effective dose of medicament to a user. Various methods of forming uniform films, as well as various polymers, additives and fillers, may be used, including those methods and materials described in U.S. Pat. Nos. 7,425,292, 7,357,891, 7,666,337, 7,824,588 and 7,897,080, which are herein incorporated by reference in their entireties. Any number of active components or pharmaceutical agents may be included in the films discussed herein. Various combinations of active components may be used in the same film product to provide a desired effect. The active component(s) may be disposed within any layer of film products formed herein or they may be placed onto one or more surfaces of the film products.

The products described herein include at least two different pharmaceutically active components, a first pharmaceutically active component and a second pharmaceutically active component. The first pharmaceutically active component may be desirably a pain-relieving component, which will be described in greater detail below, and the second pharmaceutically active component may be desirably an anti-nausea and/or vomiting component, which will also be described in greater detail below. It is particularly desirable that the first pharmaceutically active component be released from the product quickly and absorbed into the skin of the user, such as in the sublingual or buccal region of the user. In some embodiments, the product may be substantially fully dissolved within about thirty seconds. By “substantially dissolved”, it is intended that at least 90% of the dissolvable materials in the product are dissolved, and more desirably at least about 95% of the dissolvable materials in the product are dissolved. The desired effect is a fast onset of anti-pain medication, which may be measured by plasma levels. For example, there may be desirably a noticeable level of anti-pain medication (i.e., the first pharmaceutically active component) in the blood plasma of the user within about 15 minutes after placement in the user's mouth. Desirably, the level of pain experienced by a user may be reduced within about 15 minutes after placement of the product within the mouth of the user, as measured, for example, by reaching of the desired therapeutic level or by the drop in the level of the anti-pain medication (i.e., the first pharmaceutically active component) in the blood plasma of the user.

At the same time, the product desirably releases the second pharmaceutically active component at a slower rate, and in such a manner that the second pharmaceutically active component may be released into the gastrointestinal tract of the user. Thus, while the first pharmaceutically active component may be absorbed into the mucosal surface of the user, the second pharmaceutically active component may be swallowed by the user. This may be achieved, for example, by using a multi-layered product, where the first layer includes the first pharmaceutically active component and may be maintained in contact with the mucosal surface of the user, and the second layer includes the second pharmaceutically active component, which may be maintained out of contact with the mucosal surface of the user. In such embodiments, a multi-layered film product may be quite useful. Specifically, a multi-layered film product where the first layer (including the first pharmaceutically active component) is mucoadhesive, may be useful.

In some embodiments, the first and second pharmaceutically active components are chemically reactive with each other, and thus should be kept physically separate from each other during the manufacture stage and also while in the dosage product. It may be desired that the first pharmaceutically active component be maintained in a first domain within the product, and the second pharmaceutically active component be maintained within a second domain within the product. If the first pharmaceutically active component is acidic in nature, the first domain may likewise be acidic or may be neutral. If the second pharmaceutically active component is basic in nature, the second domain may likewise be basic or may be neutral. If both domains are neutral, a single layer product may be more easily prepared.

In some embodiments, a multi-layered product may be also quite useful, since the first pharmaceutically active component may be in the first layer and the second pharmaceutically active component may be in the second layer. Alternatively, one or both of the pharmaceutically active components may be encapsulated in a protective material, such as a polymer or combination of polymers, as described below. The first or second pharmaceutically active component may be encapsulated, so as to control its release. In addition, the product may also have inclusion complexes, coatings, or ion exchange resins so as to prevent premature reaction between actives, particularly if more than one active is in the same layer and may potentially cause stability issues or loss of activity or ability to be released, adsorbed or absorbed by the body. Such modifiers may be used to achieve a desired release amount or rate, or may be used to achieve a desired absorption, adsorption or uptake by the user.

The first pharmaceutically active component is desirably an anti-pain medication, which may be useful in relieving the pain felt by a user. The pain to be treated may include, for example, a headache experienced by the user. Pharmaceutical active components useful in treating pain associated with a migraine or other central nervous system related pain disorder include, but are not limited to, triptans, such as sumatriptan, zolmitriptan, rizatriptan, eletriptan, naratriptan, almotriptan, and frovatriptan. Other components that may be used to treat pain include, but are not limited to, Inderal (propranolol), Blocadren (timolol), Toprol (metoprolol), Corzide (nadolol), Dihydroergotamine, Prochlorperazine, Depakene (sodium valproate), Depakote (divalproex sodium), Isoptin (Verapamil), Nimotop (Nimodipine), Elavil, Sinequan, Norpramin, Prozac, Zoloft, and Paxil. Non-limiting examples of non-migraine analgesics include aspirin and Nonsteroidal anti-inflammatory drugs (“NSAIDS” (e.g., Celecoxib (Celebrex®), Diclofenac (Cataflam®, Voltaren®, Arthrotec®) Diflunisal (Dolobie), Etodolac (Lodine®), Fenoprofen (Nalfon®), Flurbirofen (Ansaid®), Ibuprofen (Motrin®, ADVIL.®, NUPRIN®, TabProfen®, Vicoprofen®, Combunox®), Indornethacin (Indocin®, Indo-Lemmon®, Indornethagan®), Ketoprofen (Oruvail®), Ketorolac (Toradol®), Mefenamic acid (Ponstel®, commercially available First Horizon Pharmaceutical), flufenamic acid ([N-(3-trifluoromethylphenyl)anthranilic acid]), Meloxicam (Mobic®), Naburnetone (Relafen®), Naproxen (Naprosyn®, ALEVE®, Anaprox®, Naprelan®, Naprapac®), Oxaprozin (Daypro®), Piroxicam (Feldene), Sulindac (Clinoril®) and Tolmetin (Tolectin®)) Any pain reliever may be used alone or in combination with any other components that may relieve pain in a user.

The second pharmaceutically active component is desirably an anti-nausea and/or anti-vomiting medication, which may be useful in relieving the feeling of nausea and/or anti-vomiting experienced by a user. Pharmaceutical active components useful in treating nausea and/or vomiting in an individual include, but are not limited to, 5-HT₃ receptor antagonists, Dopamine antagonists, NK1 receptor antagonists, antihistamines (H₁ histamine receptor antagonists), cannabinoids, benzodiazepines, anticholinergics, steroids, trimethobenzamide, ginger-based materials, and emetrol. Any of these components may be used alone or in combination with other components useful for treating nausea and/or vomiting.

5-HT₃ receptor antagonists block serotonin receptors in the central nervous system and gastrointestinal tract. As such, they can be used to treat post-operative and cytotoxic drug nausea and/or vomiting. Suitable 5-HT₃ receptor antagonists include, for example, Dolasetron (Anzemet™), Granisetron (Kytril™, Sancuso™), Ondansetron (Zofran™), Tropisetron (Navoban™), Palonosetron (Aloxi™), and Mirtazapine (Remeron™).

Dopamine antagonists act in the brain and are used to treat nausea and/or vomiting associated with neoplastic disease. Suitable dopamine antagonists include, but are not limited to, Domperidone, Olanzapine, Droperidol, haloperidol, chlorpromazine, promethazine, prochlorperazine, Alizapride, metoclopramide, and Prochlorperazine (Compazine™, Stemzine™, Buccastem™, Stemetil™, Phenotil™).

Suitable NK1 receptor antagonist include, but are not limited to, Aprepitant (Emend™) and Casopitant Investigational NK1 receptor antagonist.

Antihistamines (also referred to as H₁ histamine receptor antagonists) are effective in many conditions, including motion sickness, morning sickness in pregnancy, and to combat opioid nausea and/or vomiting. Suitable antihistamines include, but are not limited to, Cyclizine, Diphenhydramine (Benadryl), Dimenhydrinate (Gravol™, Dramamine™), Doxylamine, Meclozine (Bonine™, Antivert™), and Hydroxyzine.

Cannabinoids are used in patients with cachexia, cytotoxic nausea and/or vomiting, or who are unresponsive to other agents. Suitable cannabinoids include, but are not limited to, Cannabis (Medical marijuana, which is a Schedule I drug in the United States), Dronabinol (Marinol™, which is a Schedule III drug in the United States), and may include some synthetic cannabinoids such as Nabilone (Cesamet) or the JWH series.

Suitable Benzodiazepines include, but are not limited to, Midazolam and Lorazepam. Suitable Anticholinergics include, for example, Hyoscine (also known as scopolamine). Suitable Steroids include, for example, Dexamethasone (Decadron™).

The product desirably includes a pharmaceutically active level of the first pharmaceutically active component (an anti-pain medication) and a pharmaceutically active level of the second pharmaceutically active component (an anti-nausea and/or vomiting medication). The first and second pharmaceutically active components may be interspersed throughout the same layer of a product, such as a tablet, pill, film or patch. Alternatively, a multi-layered product may be used, with each of the first and second pharmaceutically active components maintained in separate layers. The first layer may be a tablet, pill, film or patch, and the second layer may be a tablet, pill, film or patch, being secured to the first layer. Desirably, the first layer may be a film and the second layer may be also a film, which is laminated to the first layer.

In one particular method of forming a desired film product, the product may be a multi-layered film, including a first pharmaceutical active component in the first layer and a second pharmaceutical active component in a second layer. In some embodiments, a first film structure is first formed, which will form the first layer. A film-forming material or matrix is first formed, using polymers, solvents, fillers, and the like as desired, to provide a film product having the desired dissolution or disintegration time. In some aspects, this first layer may be formed through the use of a combination of polymers, such as polyethylene oxide and cellulose, such as hydroxypropylmethylcellulose (“HPMC”). The film-forming matrix is deposited onto a substrate and then dried to form a film product. Drying may be achieved through any desired suitable method This first layer includes a desired level of the first pharmaceutical active component dispersed throughout.

In multi-layered films, a second film-forming material, which will be used to form the second layer, will also be prepared. Desirably, this second material includes a second pharmaceutical active component, and will be used to form a film product having the desired dissolution or disintegration rates described above. In preferred aspects, the dissolution or disintegration rate of the second material (and subsequently second layer) may be slower than that of the first material (and subsequently first layer). The second film layer may be deposited onto the first layer and dried, or may be formed and dried separately, and then the first and second layers secured together, such as through lamination or chemical bonding.

In some embodiments, the film product may be a single layered film, with the first and second pharmaceutical active components each dispersed throughout the film. If the first and second pharmaceutical active components are reactive with each other, then the two components may be kept physically separate from each other, such as by surrounding one or both first and second pharmaceutical active components with a non-reactive material, such as a polymer or taste masking component. For example, one of the pharmaceutically active components may be basic in nature, and thus may be surrounded with a taste masking material such as sodium bicarbonate, or the like.

The process of forming a film product may include depositing a wet film matrix onto the surface of a substrate. Any desired substrate may be used, including, for example, mylar, paper, plastic, metal, foil, and combinations thereof. The substrate may be laminated if desired. Further, the substrate may be chemically treated on one or more surfaces prior to depositing the wet film matrix thereon. Desirably, the substrate is substantially flat, but is flexible to allow for rolling, such as for storage or for packaging of the formed film products. The substrate may include one or more dams, such as that disclosed in Applicant's co-pending U.S. patent application Ser. No. 12/711,883, filed Feb. 24, 2010, the entire contents of which are incorporated by reference herein. In some embodiments, the substrate is an already-formed film layer, which includes the first pharmaceutically active component, and the wet film deposited thereon includes the second pharmaceutically active component.

The film product, whether a single-layer or multi-layer film, may include various film-forming components, including polymers and solvents, in addition to any optional components such as flavors, colors, sweeteners and the like. The polymer or polymers used in each domain may be the same or they may be different. If they are different, the polymers should be compatible with each other to the extent they can adhere to each other or physically intertwine, chemically bond, complex, solvent bond, or interact with each other by such similar methods. In some aspects, the first film forming components may include polymers selected from the group consisting of polyethylene oxide, cellulose derivatives, and combinations thereof. The polyethylene oxide may have a molecular weight of from about 100,000 to about 900,000, or may be higher. Polymers with higher molecular weights (e.g. up to about 7 million) may be useful. In some aspects, the film may include polyethylene oxide of higher molecular weight (from about 600,000 to about 900,000) and lower molecular weight (from about 100,000 to about 300,000) in combination.

Preferred cellulose derivatives include, for example, methylcellulose and (hydroxypropyl methylcellulose, “HPMC”), and derivatives thereof. Other polymers such as polyvinylpyrrolidinone (“PVP”) as well as combination of PVP and vinyl acetate (“PVP/VA”) may also be used. The polymer(s) in the film product, whether a single layer or multi-layer product, as well as their molecular weights are desirably chosen so as to provide a desired slower dissolution or disintegration time, as explained above. The differences in the rate of dissolution or disintegration between the first and second layers can also be adjusted by selecting suitable particular solvent(s).

In addition to film-forming polymer(s), the film product may include one or more solvents. The solvent or solvents in each layer may be the same or they may be different. Useful solvents include, but are not limited to, polar solvents such as water. Non-polar solvents may be used if desired. Combinations of polar and non-polar solvents are also useful. In addition, the amount of solvent or solvents in each layer may be the same or they may be different. Non-limiting examples of useful solvents may include water, ethanol, propanol acetone and combinations thereof. During the formation of the product, that is, prior to any drying steps, there may be at least 15% solvent by weight of the film-forming material. In some aspects, more solvent may be used, and thus each domain may have up to around 70% by weight solvent.

Other materials may be included in the film-forming materials, such as sweeteners, flavors, colors, fillers, and the like. For example, one or both layers may include sugar or sugar-free sweeteners, such as polyols.

As discussed above, the active components of the present invention may be provided in the form of a film dosage form. In such embodiments, a flowable film-forming matrix is prepared to be uniform in content in accordance with the teachings of the present invention. Uniformity should be maintained as the flowable viscoelastic mass is formed into a film and dried. During the drying process of the present invention, several factors produce uniformity within the film while maintaining the matrix temperature of the film at a temperature where the degradation of active/s is eliminated or minimized so that suitable potency ranges can be maintained. First, the films of the present invention have an extremely short heat history, usually only on the order of minutes, so that total temperature exposure is minimized to the extent possible. The films are controllably dried to prevent aggregation and migration of components, as well as preventing heat build up within. The films may be dried from the bottom. In any drying method, however, it is desirable to rapidly form a visco-elastic film within the first fifteen minutes of drying, and desirably within the first ten minutes of drying, and even more preferably within the first four minutes of drying. Due to the short heat exposure and evaporative cooling, the film components such as drug or volatile actives remain minimally affected by high temperatures, and small-scale particles of active agent are maintained in a non-aggregated fashion. In contrast, skinning on the top surface traps liquid carrier molecules of increased energy within the film, thereby causing the temperature within the film to rise and exposing active components to high, potentially deleterious temperatures. Preferably, the interior of the film does not reach a level at which degradation of the active contained therein will occur or, if occurring, the degradation does not affect the potency of the film. Once the rapid formation of a visco-elastic film is achieved, to “lock-in” the uniformity of active content per unit dose, the film may be further dried, such as by exposure to heat, radiation, or other drying sources. The step of further drying the thus-formed visco-elastic film may reduce the water or solvent content in the film to less than 12% by weight, less than 10% by weight, less than 8% by weight, less than 6% by weight, less than 4% by weight, or less than 2% by weight.

Second, thermal mixing occurs within the film due to controlled drying and absence of surface skinning. Thermal mixing occurs via convection currents in the film. As heat is applied to the bottom of the film, the liquid near the bottom increases in temperature, expands, and becomes less dense. As such, this hotter liquid rises and cooler liquid takes its place. While rising, the hotter liquid mixes with the cooler liquid and shares thermal energy with it, i.e., transfers heat. As the cycle repeats, thermal energy is spread throughout the film.

Robust thermal mixing achieved by the controlled drying process of the present invention produces uniform heat diffusion throughout the film. In the absence of such thermal mixing, “hot spots” may develop. Pockets of heat in the film result in the formation of particle aggregates or danger areas within the film and subsequent non-uniformity. The formation of such aggregates or agglomerations is undesirable because it leads to non-uniform films in which the active may be randomly distributed. Such uneven distribution may lead to large differences in the amount of active per film, which is problematic from a safety and efficacy perspective.

Furthermore, thermal mixing helps to maintain a lower overall temperature inside the film. Although the film surfaces may be exposed to a temperature above that at which the active component degrades, the film interior may not reach this temperature. Due to this temperature differential, the active does not degrade to a level that reduces the amount of viable active to an undesirable amount. That is, while some degradation of the active may occur during drying, the remaining active is within about 10% of a target level or potency of the active, as will be explained below.

For instance, the films of the present invention may be dried for 20 minutes or less, 15 minutes or less, desirably 10 minutes or less to achieve a desired solvent content. Drying the films at 80° C. for 10 minutes produces a temperature differential of about 5° C. This means that after 10 minutes of drying, the temperature of the inside of the film is 5° C. less than the outside exposure temperature. In many cases, however, drying times of less than 10 minutes are sufficient, such as 4 to 6 minutes. Drying for 4 minutes may be accompanied by a temperature differential of about 30° C., and drying for 6 minutes may be accompanied by a differential of about 25° C. Due to such large temperature differentials, the films may be dried at efficient, high external temperatures without causing heat sensitive actives to degrade. Further drying may be used to reduce the solvent content to an even lower level.

After mechanical mixing, the film may be placed on a conveyor for continued thermal mixing during the drying process. At the outset of the drying process, the film preferably is heated from the bottom as it is travels via conveyor. Heat may be supplied to the film by a heating mechanism, such as, but not limited to, a dryer. As the film is heated, the liquid carrier, or volatile, begins to evaporate. Thermal mixing also initiates as hotter liquid rises and cooler liquid takes its place. Because no skin forms on the top surface of the film, the volatile liquid continues to evaporate and thermal mixing continues to distribute thermal energy throughout the film. Once a sufficient amount of the volatile liquid has evaporated, thermal mixing has produced uniform heat diffusion throughout the film. The components desirably are locked into a uniform distribution throughout the film. It may be desired to form a visco-elastic solid rapidly, for example within the first 15 minutes or less, desirably within the first 10 minutes or less, more desirably within the first 6 minutes or less, and most desirably within the first 0.5 minutes to 4 minutes to lock-in the active(s). Although minor amounts of liquid carrier, i.e., water, water/alcohol carrier, or other suitable carrier, may remain subsequent to formation of the visco-elastic film, the film may be dried further without affecting the desired uniformity of active content and heterogeneity of the film, if desired. Further drying forms the final film, by desirably removing solvent from the visco-elastic solid such that less than 12% of solvent remains, less than 10% of solvent remains, and more desirably less than 8% of solvent remains, and most desirably less than 6% of the solvent remains in the final film.

The internal temperature of the film matrix during drying is desirably less than about 100° C., desirably less than about 70° C., less than about 60° C., less than about 50° C., less than about 40° C., or less than about 30° C. The external temperature at which the film is dried may be higher than the internal temperature, and may be, for example, 40° C. or greater, 50° C. or greater, 60° or greater, 70° C. or greater, may be 80° C. or greater, or may be 100° C. or greater. The film may be exposed to a high temperature, such as about 100° C. or greater, for a short period of time, such as less than about a few minutes. For example, the air temperatures used to dry the film may be about 130° C. or higher, the upper limit being dictated by the specific formulation (e.g., the types and amount of solvent, polymers, fillers, etc.) and active used. The air temperature is also dictated by the length of the drying required to rapidly form the visco-elastic film to lock in the uniformity of content, as discussed herein.

Furthermore, particles or particulates may be added to the film-forming composition or material after the composition or material is cast into a film. For example, particles may be added to the film prior to the drying of the film. Particles may be controllably metered to the film and disposed onto the film through a suitable technique, such as through the use of a doctor blade, which is a device which marginally or softly touches the surface of the film and controllably disposes the particles onto the film surface. Other suitable, but non-limiting, techniques include the use of an additional roller to place the particles on the film surface, spraying the particles onto the film surface, and the like. The particles may be placed on either or both of the opposed film surfaces, i.e., the top and/or bottom film surfaces. Desirably, the particles are securably disposed onto the film, such as being embedded into the film. Moreover, such particles are desirably not fully encased or fully embedded into the film, but remain exposed to the surface of the film, such as in the case where the particles are partially embedded or partially encased.

Monitoring and control of the thickness of the film also contributes to the production of a uniform film by providing a film of uniform thickness. The thickness of the film may be monitored with gauges such as Beta Gauges. A gauge may be coupled to another gauge at the end of the drying apparatus, i.e. drying oven or tunnel, to communicate through feedback loops to control and adjust the opening in the coating apparatus, resulting in control of uniform film thickness. Alternatively, the thickness of the film can also be controlled by manual measurement during the production process to achieve the desired thickness of the film.

The film products are generally formed by combining a properly selected polymer and polar solvent, as well as any agent or filler as desired. Desirably, the solvent content of the combination is at least about 30% by weight of the total combination. The material formed by this combination is formed into a film, desirably by roll coating, and then dried, desirably by a rapid and controlled drying process to maintain the uniformity of the film, more specifically, a non-self-aggregating uniform heterogeneity. The resulting film will desirably contain less than 12% by weight solvent, less than about 10% by weight solvent, more desirably less than about 8% by weight solvent, even more desirably less than about 6% by weight solvent and most desirably less than about 4%. The solvent may be water, a polar organic solvent including, but not limited to, ethanol, isopropanol, acetone, methylene chloride, or any combination thereof.

Consideration of the above discussed parameters, such as, but not limited to, rheology properties, viscosity, mixing method, casting method and drying method, also impact material selection for the different components of the present invention. Furthermore, such consideration with proper material selection provides the compositions of the present invention, including a pharmaceutical and/or cosmetic dosage form or film product having no more than a 10% variance of a pharmaceutical and/or cosmetic active per unit volume, or no more of than a ten percent (10%) variance by weight of an active-carrying component (e.g. nanoparticles) per unit volume of the film product. The compositional uniform distribution may be measured by preparing substantially equally-sized individual unit doses from the film, where the substantially equally-sized individual unit doses do not vary from each other by more than 10% of active component.

In other words, the uniformity of the present invention may be determined by the presence of no more than a 10% by weight of pharmaceutical, biological, bioeffecting, active-containing component, and/or cosmetic variance throughout the matrix, or in other words, substantially equally sized dosage units cut from the same film do not vary from each other by more than about 10% of the target level of active content. Desirably, the variance is less than 5% by weight, less than 2% by weight, less than 1% by weight, or less than 0.5% by weight.

In some embodiments, compositional uniformity may be measured with respect to a target or desired level of active. The film is prepared so as to provide each unit dose with a target level of active therein. Compositional uniformity is achieved when each individual unit dose varies by no more than 10% of the target level of active (by weight). More desirably, each unit dose varies by no more than 8% of the target level of active, no more than 6% of the target level of active, or no more than 4% of the target level of active. In addition, if any degradation of the active occurs during the process, the amount of remaining active that has not degraded should be within 10% of the target level, or within about 8% of the target level, or within about 6% of the target level, or within about 4% of the target level. The target level can be, for example, what is described on the label of the product.

### Film-Forming Polymers

The film units of the present invention include at least one water soluble polymer. The films may also include water swellable or water insoluble polymers, if desired.

In some embodiments, the self-supporting film includes a saccharide-based polymer, which is water soluble. For example, the saccharide-based polymer may be cellulose or a cellulose derivative. Specific examples of useful saccharide-based, water soluble polymers include, but are not limited to, polydextrose, pullulan, hydroxypropylmethyl cellulose (HPMC), hydroxyethyl cellulose (HPC), hydroxypropyl cellulose, carboxymethyl cellulose, sodium alginate, xanthan gum, tragancanth gum, guar gum, acacia gum, arabic gum, starch, gelatin, and combinations thereof.

In some preferred embodiments, the saccharide-based polymer may be at least one cellulosic polymer, polydextrose, or combinations thereof. The film may also include non-saccharide-based, water soluble or water insoluble polymers. Examples of non-saccharide based, water soluble polymers include polyethylene oxide, polyvinylpyrrolidone, polyvinyl alcohol, polyethylene glycol, polyacrylic acid, methylmethacrylate copolymer, carboxyvinyl copolymers, and combinations thereof. Specific examples of useful water insoluble polymers include, but are not limited to, ethyl cellulose, hydroxypropyl ethyl cellulose, cellulose acetate phthalate, hydroxypropyl methyl cellulose phthalate and combinations thereof.

In some further preferred embodiments, the polymer is a combination of hydroxypropylmethyl cellulose and polyethylene oxide. In some other preferred embodiments, the polymer is a combination of polydextrose and polyethylene oxide. In still further preferred embodiments, the polymer is a combination of polydextrose, hydroxy propylmethyl cellulose and polyethylene oxide.

As used herein, the phrase “water soluble polymer” and variants thereof refer to a polymer that is at least partially soluble in water, and desirably fully or predominantly soluble in water, or absorbs water. In some embodiments, the film unit of the present invention is at least partially dissolvable when exposed to a wetting agent. In some other embodiments, the inventive film unit is substantially dissolvable when exposed to a wetting agent.

Polymers that absorb water are often referred to as being water swellable polymers. The materials useful with the present invention may be water soluble or water swellable at room temperature and other temperatures, such as temperatures exceeding room temperature. Moreover, the materials may be water soluble or water swellable at pressures less than atmospheric pressure. Desirably, the water soluble polymers are water soluble or water swellable having at least 20 percent by weight water uptake. Water swellable polymers having a 25 or greater percent by weight water uptake are also useful. Films or dosage forms of the present invention formed from such water soluble polymers are desirably sufficiently water soluble to be dissolvable upon contact with bodily fluids.

Other polymers useful for incorporation into the films of the present invention include biodegradable polymers, copolymers, block polymers and combinations thereof. Among the known useful polymers or polymer classes which meet the above criteria are: poly(glycolic acid) (PGA), poly(lactic acid) (PLA), polydioxanoes, polyoxalates, poly(a-esters), polyanhydrides, polyacetates, polycaprolactones, poly(orthoesters), polyamino acids, polyaminocarbonates, polyurethanes, polycarbonates, polyamides, poly(alkyl cyanoacrylates), and mixtures and copolymers thereof. Additional useful polymers include, stereopolymers of L- and D-lactic acid, copolymers of bis(p-carboxyphenoxy)propane acid and sebacic acid, sebacic acid copolymers, copolymers of caprolactone, poly(lactic acid)/poly(glycolic acid)/polyethyleneglycol copolymers, copolymers of polyurethane and (poly(lactic acid), copolymers of polyurethane and poly(lactic acid), copolymers of α-amino acids, copolymers of α-amino acids and caproic acid, copolymers of α-benzyl glutamate and polyethylene glycol, copolymers of succinate and poly(glycols), polyphosphazene, polyhydroxy-alkanoates and mixtures thereof. Binary and ternary systems are contemplated.

Other specific polymers useful include those marketed under the Medisorb and Biodel trademarks. The Medisorb materials are marketed by the Dupont Company of Wilmington, Del. and are generically identified as a “lactide/glycolide co-polymer” containing “propanoic acid, 2-hydroxy-polymer with hydroxy-polymer with hydroxyacetic acid.” Four such polymers include lactide/glycolide 100L, believed to be 100% lactide having a melting point within the range of 338°-347° F. (170°-175° C.); lactide/glycolide 100L, believed to be 100% glycolide having a melting point within the range of 437°-455° F. (225°-235° C.); lactide/glycolide 85/15, believed to be 85% lactide and 15% glycolide with a melting point within the range of 338°-347° F. (170°-175° C.); and lactide/glycolide 50/50, believed to be a copolymer of 50% lactide and 50% glycolide with a melting point within the range of 338°-347° F. (170°-175° C.).

The Biodel materials represent a family of various polyanhydrides which differ chemically.

Although a variety of different polymers may be used, it is desired to select polymers to provide a desired viscosity of the mixture prior to drying. For example, if the agent or other components are not soluble in the selected solvent, a polymer that will provide a greater viscosity is desired to assist in maintaining uniformity. On the other hand, if the components are soluble in the solvent, a polymer that provides a lower viscosity may be preferred.

The polymer plays an important role in affecting the viscosity of the film. Viscosity is one property of a liquid that controls the stability of the topical agent in a solution, an emulsion, a colloid or a suspension. Generally the viscosity of the matrix will vary from about 400 cps to about 100,000 cps, preferably from about 800 cps to about 60,000 cps, and most preferably from about 1,000 cps to about 40,000 cps. Desirably, the viscosity of the film-forming matrix will rapidly increase upon initiation of the drying process.

The viscosity may be adjusted based on the selected topical agent component, depending on the other components within the matrix. For example, if the component is not soluble within the selected solvent, a proper viscosity may be selected to prevent the component from settling which would adversely affect the uniformity of the resulting film. The viscosity may be adjusted in different ways. To increase viscosity of the film matrix, the polymer may be chosen of a higher molecular weight or crosslinkers may be added, such as salts of calcium, sodium and potassium. The viscosity may also be adjusted by adjusting the temperature or by adding a viscosity increasing component. Components that will increase the viscosity or stabilize the emulsion/suspension include higher molecular weight polymers and polysaccharides and gums, which include without limitation, alginate, carrageenan, hydroxypropyl methyl cellulose, locust bean gum, guar gum, xanthan gum, dextran, gum arabic, gellan gum and combinations thereof.

It has also been observed that certain polymers which when used alone would ordinarily require a plasticizer to achieve a flexible film, can be combined without a plasticizer and yet achieve flexible films. For example, HPMC and HPC when used in combination provide a flexible, strong film with the appropriate plasticity and elasticity for manufacturing and storage. No additional plasticizer or polyalcohol is needed for flexibility.

Additionally, polyethylene oxide (PEO), when used alone or in combination with a hydrophilic cellulosic polymer and/or polydextrose, achieves flexible, strong films. Additional plasticizers or polyalcohols are not needed for flexibility. Non-limiting examples of suitable cellulosic polymers for combination with PEO include HPC and HPMC. PEO and HPC have essentially no gelation temperature, while HPMC has a gelation temperature of 58-64° C. (Methocel EF available from Dow Chemical Co.). Moreover, these films are sufficiently flexible even when substantially free of organic solvents, which may be removed without compromising film properties. As such, if there is no solvent present, then there is no plasticizer in the films. PEO based films also exhibit good resistance to tearing, little or no curling, and fast dissolution rates when the polymer component contains appropriate levels of PEO.

To achieve the desired film properties, the level and/or molecular weight of PEO in the polymer component may be varied. Modifying the PEO content affects properties such as tear resistance, dissolution rate, and adhesion tendencies. Thus, one method for controlling film properties is to modify the PEO content. For instance, in some embodiments rapid dissolving films are desirable. By modifying the content of the polymer component, the desired dissolution characteristics can be achieved.

In accordance with the present invention, PEO desirably ranges from about 20% to 100% by weight in the polymer component. In some embodiments, the amount of PEO desirably ranges from about 1 mg to about 200 mg. The hydrophilic cellulosic polymer and/or polydextrose ranges from about 0% to about 80% by weight, or in a ratio of up to about 4:1 with the PEO, and desirably in a ratio of about 1:1.

In some embodiments, it may be desirable to vary the PEO levels to promote certain film properties. To obtain films with high tear resistance and fast dissolution rates, levels of about 50% or greater of PEO in the polymer component are desirable. To achieve adhesion prevention, i.e., preventing the film from adhering to the roof of the mouth, PEO levels of about 20% to 75% are desirable. In some embodiments, however, adhesion to the roof of the mouth may be desired, such as for administration to animals or children. In such cases, higher levels of PEO may be employed. More specifically, structural integrity and dissolution of the film can be controlled such that the film can adhere to mucosa and be readily removed, or adhere more firmly and be difficult to remove, depending on the intended use.

The molecular weight of the PEO may also be varied. High molecular weight PEO, such as about 4 million, may be desired to increase mucoadhesivity of the film. More desirably, the molecular weight may range from about 100,000 to 900,000, more desirably from about 100,000 to 600,000, and most desirably from about 100,000 to 300,000. In some embodiments, it may be desirable to combine high molecular weight (600,000 to 900,000) with low molecular weight (100,000 to 300,000) PEOs in the polymer component.

For instance, certain film properties, such as fast dissolution rates and high tear resistance, may be attained by combining small amounts of high molecular weight PEOs with larger amounts of lower molecular weight PEOs. Desirably, such compositions contain about 60% or greater levels of the lower molecular weight PEO in the PEO-blend polymer component.

To balance the properties of adhesion prevention, fast dissolution rate, and good tear resistance, desirable film compositions may include about 50% to 75%, by weight of the total composition, low molecular weight PEO, optionally combined with a small amount of a higher molecular weight PEO, with the remainder of the polymer component containing a hydrophilic cellulosic polymer (HPC or HPMC) and/or polydextrose.

In some embodiments the film may include polyvinyl alcohol (PVA), alone or in combination with at least one additional polymer Examples of an additional polymer include a cellulosic polymer, starch, polyvinyl pyrrolidone (PVP), polyethylene oxide (PEO), an alginate, a pectin, or combinations thereof. PVA can be used in the films to improve film strength and/or to vary and slow dissolution times. The films are especially useful for the delivery of cosmetics, nutraceuticals and pharmaceuticals. In a preferred embodiment, the film includes PVA without any added plasticizers. For example, the film can include both PVA, which provides strength to the film and PEO, which provides flexibility to the film and nay obviate the need for a plasticizer.

PVA can be used in varying amounts depending upon the product application and characteristics desired. For example, in general, a larger amount of PVA will increase film strength and increase dissolution time. For films that require high active dosing, PVA can be used effectively at minimum amount of 0.5, preferably 1%, more preferably 5%, by weight of the film, to improve film strength. The PVA an be effectively used at a maximum amount, for example, 80%, preferably 50%, more preferably 25% by weight of the film. For slowing dissolution time, PVA can be used at levels as high as 80%. A film containing an active can be coated on one or both surfaces with a PVA containing layer to modify the dissolution of the film and the release of an active from the film.

High loading of actives can decrease the strength and flexibility of the film. Including PVA in the film either alone or in combination with at least one other polymer can increase the tensile strength of the film. Also, drug particles or taste-masked or coated or modified release drug particles may have a larger particle size, which can make loading of these particles into the film difficult. PVA can increase the viscosity of the film solution to allow improved drug loading.

### Controlled Release Films

The term “controlled release” is intended to mean the release of the components at a pre-selected or desired rate. For example, in embodiments where the film includes nanoparticles within the body of the film, it may be desirable to control its release from the film. This rate will vary depending upon the application. Desirable rates include fast or immediate release profiles as well as delayed, sustained or sequential release. Combinations of release patterns, such as initial spiked release followed by lower levels of sustained release of active are contemplated. Pulsed releases of the agent are also contemplated.

Dissolvable films generally fall into three main classes: fast dissolving, moderate dissolving and slow dissolving. Films of the present invention are dissolvable in the presence of liquid, such as in the oral cavity of the user or when mixed with a liquid, such as water. Fast dissolving films generally dissolve in about 1 second to about 30 seconds. Moderate dissolving films generally dissolve in about 1 to about 30 minutes, and slow dissolving films generally dissolve in more than 30 minutes, e.g., up to about 60 minutes or more. Fast dissolving films may consist of low molecular weight hydrophilic polymers (i.e., polymers having a molecular weight between about 1,000 to 200,000). In contrast, slow dissolving films generally have high molecular weight polymers (i.e., having a molecular weight in the millions).

Moderate dissolving films tend to fall in between the fast and slow dissolving films. Moderate dissolving films dissolve rather quickly, but also have a good level of mucoadhesion. Moderate films are also flexible, quickly wettable, and are typically non-irritating to the user. For oral-dissolving films, moderate dissolving films are preferred, since such films provide a quick enough dissolution rate (between about 1 minute and about 5 minutes), while providing an acceptable mucoadhesion level such that the film is not easily removable once it is placed in the oral cavity of the user.

The polymers that are chosen for the films of the present invention may also be chosen to allow for controlled disintegration of the components. This may be achieved by providing a substantially water insoluble film that incorporates a nanoparticle that will be released from the film over time. This may be accomplished by incorporating a variety of different soluble or insoluble polymers and may also include biodegradable polymers in combination. Alternatively, coated controlled release agent particles may be incorporated into a readily soluble film matrix to achieve the controlled release property of the nanoparticles.

The convenience of administering a single dose of a medication which releases components in a controlled fashion over an extended period of time, as opposed to the administration of a number of single doses at regular intervals has long been recognized in the pharmaceutical arts. The advantage to the patient and clinician in having consistent and uniform levels of medication delivered to the body over an extended period of time are likewise recognized.

In some embodiments, the erosion or disintegration of the film (e.g., the residence time) can be controlled by a combination of factors. One factor may be the thickness of the film, whereby due to its physical dimensions, disintegration of a thicker film in the body, such as in the oral cavity, as with a buccal dosage form, is designed to be slower than a film that has thinner dimensions. Additionally, the selection of amounts and types of polymers and/or molecular weights of polymers, as well as inclusion of additives or disintegration aides, may be employed to vary residence time. Selection of polymers and inclusion of additives may be used alone or in combination with the use of different thicknesses to achieve the desired residence time. These factors have the ability to effect the release of active in a desired time.

### Optional Components

A variety of other components and fillers may also be added to the films of the present invention. These may include, without limitation, surfactants; plasticizers which assist in compatibilizing the components within the mixture; polyalcohols; anti-foaming agents, such as silicone-containing compounds, which promote a smoother film surface by releasing oxygen from the film; and thermo-setting gels such as pectin, carageenan, and gelatin, which help in maintaining the dispersion of components.

The variety of additives that can be incorporated into the inventive compositions may provide a variety of different functions. Examples of classes of additives include excipients, lubricants, buffering agents, stabilizers, blowing agents, pigments, coloring agents, fillers, bulking agents, fragrances, release modifiers, adjuvants, plasticizers, flow accelerators, mold release agents, polyols, granulating agents, diluents, binders, buffers, absorbents, glidants, adhesives, anti-adherents, acidulants, softeners, resins, demulcents, solvents, surfactants, emulsifiers, elastomers and mixtures thereof. These additives may be added with the active ingredient(s).

Useful additives include, for example, gelatin, vegetable proteins such as sunflower protein, soybean proteins, cotton seed proteins, peanut proteins, grape seed proteins, whey proteins, whey protein isolates, blood proteins, egg proteins, acrylated proteins, water soluble polysaccharides such as alginates, carrageenans, guar gum, agar-agar, xanthan gum, gellan gum, gum arabic and related gums (gum ghatti, gum karaya, gum tragancanth), pectin, water soluble derivatives of cellulose: alkylcelluloses hydroxyalkylcelluloses and hydroxyalkylalkylcelluloses, such as methylcellulose, hydroxymethylcellulose, hydroxyethylcellulose, hydroxypropylcellulose, hydroxyethylmethylcellulose, hydroxypropylmethylcellulose, hydroxybutylmethylcellulose, cellulose esters and hydroxyalkylcellulose esters such as cellulose acetate phthalate (CAP), hydroxypropylmethylcellulose (HPMC); carboxyalkylcelluloses, carboxyalkylalkylcelluloses, carboxyalkylcellulose esters such as carboxymethylcellulose and their alkali metal salts; water soluble synthetic polymers such as polyacrylic acids and polyacrylic acid esters, polymethacrylic acids and polymethacrylic acid esters, polyvinylacetates, polyvinylalcohols, polyvinylacetatephthalates (PVAP), polyvinylpyrrolidone (PVP), PVY/vinyl acetate copolymer, and polycrotonic acids; also suitable are phthalated gelatin, gelatin succinate, crosslinked gelatin, shellac, water soluble chemical derivatives of starch, cationically modified acrylates and methacrylates possessing, for example, a tertiary or quaternary amino group, such as the diethylaminoethyl group, which may be quaternized if desired; and other similar polymers.

Such extenders may optionally be added in any desired amount desirably within the range of up to about 80%, desirably about 3% to 50% and more desirably within the range of 3% to 20% based on the weight of all components.

Further additives may be glidants and opacifiers, such as the oxides of magnesium aluminum, silicon, titanium, etc. desirably in a concentration range of about 0.02% to about 3% by weight and desirably about 0.02% to about 1% based on the weight of all components.

Further examples of additives are plasticizers which include polyalkylene oxides, such as polyethylene glycols, polypropylene glycols, polyethylene-propylene glycols, organic plasticizers with low molecular weights, such as glycerol, glycerol monoacetate, diacetate or triacetate, triacetin, polysorbate, cetyl alcohol, propylene glycol, sorbitol, sodium diethylsulfosuccinate, triethyl citrate, tributyl citrate, and the like, added in concentrations ranging from about 0.5% to about 30%, and desirably ranging from about 0.5% to about 20% based on the weight of the polymer.

There may further be added compounds to improve the texture of the starch material such as animal or vegetable fats, desirably in their hydrogenated form, especially those which are solid at room temperature. These fats desirably have a melting point of 50° C. or higher. Preferred are tri-glycerides with C₁₂-, C₁₄-, C₁₆-, C₁₈-, C₂₀- and C₂₂-fatty acids. These fats can be added alone without adding extenders or plasticizers and can be advantageously added alone or together with mono- and/or di-glycerides or phosphatides, especially lecithin. The mono- and di-glycerides are desirably derived from the types of fats described above, i.e. with C₁₂-, C₁₄-, C₁₆-, C₁₈-, C₂₀- and C₂₂-fatty acids.

The total amounts used of the fats, mono-, di-glycerides and/or lecithins are up to about 5% and preferably within the range of about 0.5% to about 2% by weight of the total composition

It is further useful to add silicon dioxide, calcium silicate, or titanium dioxide in a concentration of about 0.02% to about 1% by weight of the total composition. These compounds act as opacifiers and flow agents.

These additives are to be used in amounts sufficient to achieve their intended purpose. Generally, the combination of certain of these additives will alter the overall release profile of the active ingredient and can be used to modify, i.e. impede or accelerate the release.

Lecithin is one surface active agent for use in the present invention. Lecithin can be included in the feedstock in an amount of from about 0.25% to about 2.00% by weight. Other surface active agents, i.e. surfactants, include, but are not limited to, cetyl alcohol, sodium lauryl sulfate, the Spans™ and Tweens™ which are commercially available from ICI Americas, Inc. Ethoxylated oils, including ethoxylated castor oils, such as Cremophor® EL which is commercially available from BASF, are also useful. Carbowax™ is yet another modifier which is very useful in the present invention. Tweens™ or combinations of surface active agents may be used to achieve the desired hydrophilic-lipophilic balance (“HLB”). The present invention, however, does not require the use of a surfactant and films or film-forming compositions of the present invention may be essentially free of a surfactant while still providing the desirable uniformity features of the present invention.

As additional modifiers which enhance the procedure and product of the present invention are identified, Applicants intend to include all such additional modifiers within the scope of the invention claimed herein.

Other ingredients include binders which contribute to the ease of formation and general quality of the films. Non-limiting examples of binders include starches, pregelatinize starches, gelatin, polyvinylpyrrolidone, methylcellulose, sodium carboxymethylcellulose, ethylcellulose, polyacrylamides, polyvinyloxoazolidone, and polyvinylalcohols.

Films of the present invention, particularly films useful for oral ingestion by a user, may further include one or more taste-enhancing agents, such as flavors and/or sweeteners. Suitable flavors and sweeteners include those set forth in U.S. Pat. No. 7,425,292, the entire contents of which are incorporated by reference herein.

Further potential additives include solubility enhancing agents, such as substances that form inclusion compounds with active components. Such agents may be useful in improving the properties of very insoluble and/or unstable actives. In general, these substances are doughnut-shaped molecules with hydrophobic internal cavities and hydrophilic exteriors. Insoluble and/or instable actives may fit within the hydrophobic cavity, thereby producing an inclusion complex, which is soluble in water. Accordingly, the formation of the inclusion complex permits very insoluble and/or instable actives to be dissolved in water. A particularly desirable example of such agents are cyclodextrins, which are cyclic carbohydrates derived from starch. Other similar substances, however, are considered well within the scope of the present invention.

The various embodiments of the invention may include penetration and permeation enhancers. Among such useful enhancers are included medium chain mono- and diacylglycerol fatty acid derivative, such as glycerol laurate, and mixtures thereof; synthetic and natural surfactants and mixtures thereof; medium chain fatty acids and salts and esters thereof, including mono-, di- and triglycerides such as sodium caprylate and sodium caprate and mixtures thereof; bile salts; chelating agents, such as EDTA; detergents; cylodextrins, enamine derivatives, phospholipids, lecithins, cetomacrogels, sodium salicylate, sodium-5-methoxysalicyclic acid; glycerol and polyethylene glycol esters such as those sold under the name Labrasol; zonula occludens toxin; and alkyl glycosides. Additionally, combinations of penetration and permeation enhancers from different classes are also useful.

Additional permeation enhancers include, Polysorbate 80, phosphatidylcholine, nmethylpiperazine, sodium salicylate, melittin, and palmitoyl carnitine chloride (pcc). 23-lauryl ether, aprotinin, azone, benzalkonium chloride, cetylpyridinium chloride, cetyltrimethylammonium bromide, cyclodextrin, dextran sulfate, lauric acid, lauric acid/propylene glycol, lysophosphatidylcholine, menthol, methoxysalicylate, methyloleate, oleic acid, phosphatidylcholine, polyoxyethylene, sodium edta, sodium glycocholate, sodium taurocholate, sodium lauryl sulfate, sodium salicylate, sodium glycodeoxycholate, sodium taurodeoxycholate, sulfoxides, and combinations thereof.

Additional permeation and/or penetration enhancers include dimethylsulfoxide, decylmethylsulfoxide, alkysulfoxides; Alkanols, such as ethanol, propanol, butanol, pentanol, hexanol, octanolnonanol, decanol, 2-butanol, 2-pentanol, benzyl alcohol; Fatty acids and their corresponding alcohols, such as caprylic, decyl, lauryl, 2-lauryl, myristly, cetyl, stearyl oleyl, linoleyl, linolenyll alcohol; Linear carboxylic acids such as valeric, heptanoic, pelagonic, caproic, capric, lauric, Myristic, stearic, oleic, caprylic; Branched carboxylic acids such as isovaleric, neopentanoic, neoheptanoic, neononanoic, trimethyl hexanoic, neodecanoic, isostearic; fatty acid esters, such as aliphatic-isopropyl n-butyrate, isopropyl n-hexanoate, isopropyl n-decanoate, isopropyl myristate, isopropyl palmitate, octyldodecyl myristate; Alkyl esters such as ethyl acetate, butyl acetate, methyl acetate, methylvalerate, methylpropionate, diethyl sebacate, ethyl oleate; propylene glycol, polyethylene glycol, ethylene glycol, diethylene glycol, triethylene glycol, dipropylene glycol, glycerol, propanediol, butanediol, pentanediol, hexanetriol, urea, dimethylacetamide, diethyltoluamide, dimethylformamide, dimethyloctamide, dimethyldecamide; biodegradable cyclic urea, such as 1-alkyl-4-imidazolin-2-one; Pyrrolidone derivatives, such as 1-methyl-2-pyrrolidone, 2-pyrrolidone, 1-lauryl-2-pyrrolidone, 1-methyl-4-carboxy-2-pyrrolidone, 1-hexyl-4-carboxy-2-pyrrolidone, 1-lauryl-4-carboxy-2pyrrolidone, 1-methyl1-4-methoxycarbonyl-2-pyrrolidone, 1-hexyl-4-methoxycarbonyl-2 pyrrolidone, 1-lauryl-4-methoxycarbonyl-2-pyrrolidone, N-cyclohexylpyrrolidone, N-dimethylaminopropylpyrrolidone, N-cocoalkypyrrolidone, N-tallowalkylpyrrolidone; biodegradable pyrrolidone derivatives such as the fatty acid esters of N-(2-hydroxyethyl)-2-pyrrolidone; Cyclic amides such as 1-dodecylazacycloheptane-2-one (Azone), 1-geranylazacycloheptan-2-one, 1 farnesylazacycloheptan-2-one, 1-teranylgeranylazacycloheptan-2-one, 1-(3,7-dimethyloctyl)azacycloheptan-2-one, 1-(3,7,11-trimethyldodecyl)azacyclohaptan-2-one, 1-geranylazacyclohexane-2-one, 1-geranylazacyclopentan-2.5-dione, 1-farnesylazacyclopentan-2-one; Hexamethylenelauramide and its derivatives; diethanolamine, triethanolamine; Anionic surfactants such as sodium laurate, sodium lauryl sulphate; Cationic surfactants such as cetyltrimethyl ammonium bromide, tetradecyltrimethylammonium bromide, benzalkonium chloride, octadecyltrimethylammonium chloride, cetylpyridinium chloride, dodecyltrimethylammonium chloride, hexadecyltrimethylammonium chloride; Nonionic surfactants such as those sold under the trade names Poloxamer (231, 182, 184), Brij (30, 93, 96, 99), Span (20, 40, 60, 80, 85), Tween (20, 40, 60, 80), Myrj (45, 51, 52), Miglyol 840; Bile salts such as Sodium cholate, sodium salts of taurocholic, Glycholic, desoxycholic acids; lecithin; Hydrocarbons such as D-Limonene, a-pinene, B-carene; Alcohols such as a-Terpineol, terpinen-4-ol, carvol; Ketones such as carvone, pulegonee, piperitone, menthone; Oxides such as cyclohexene ocide, limonene oxide, a-pinene oxide, cyclopentene oxide, 1,8-cineole; Oils such as Ylang ylang, anise, chenopodium, eucalyptus; N-heptane, N-octane, N-nonane, N-decane, N-undecane, N-dodecane, N-tridecane, N-tetradecane, N-hexadecane; Salicylic acid and salicylates (including their methyl, ethyl, and propyl glycol derivatives); citric and succinic acid.

As previously stated, combinations of penetration and permeation enhancers from different classes are also useful.

### Forming the Film

The films of the present invention may be formed into a film strip or a sheet prior to drying. After the desired components are combined to form a multi-component matrix, including the polymer, water, and nanoparticles, as well as any other component as desired, the combination is formed into a sheet or film, by any method known in the art such as coating, spreading, casting or drawing the multi-component matrix. If a multi-layered film is desired, this may be accomplished by co-extruding more than one combination of components which may be of the same or different composition. A multi-layered film may also be achieved by coating, spreading, or casting a combination onto an already formed film layer, thus forming a multi-layered film with the already formed film layer and a second layer. The already formed film layer may be the same or may be different than the second layer. The already formed film layer may be partially dried when the second layer is coated, spread, or cast onto its surface, or it may be fully dried to a desired solvent content. The already formed film layer may be dissolvable or disintegrable, and its dissolution or disintegration time may be longer or shorter than that of the second film layer.

A number of techniques may be employed in the mixing stage to prevent bubble inclusions in the final film. To provide a composition mixture with substantially no air bubble formation in the final product, anti-foaming or surface-tension reducing agents are employed. Additionally, the speed of the mixture is desirably controlled to prevent cavitation of the mixture in a manner which pulls air into the mix. Finally, air bubble reduction can further be achieved by allowing the mix to stand for a sufficient time for bubbles to escape prior to drying the film. Desirably, the inventive process first forms a masterbatch of film-forming components without active ingredients or volatile materials. In one embodiment, the active(s) are combined with smaller mixes of the masterbatch just prior to casting. Thus, the masterbatch pre-mix can be allowed to stand for a longer time without concern for instability of the active agent or other ingredients.

Although a variety of different film-forming techniques may be used, it is desirable to select a method that will provide a flexible film, such as reverse roll coating. The flexibility of the film allows for the sheets of film to be rolled and transported for storage or prior to being cut into individual dosage forms. Desirably, the films will also be self-supporting or, in other words, able to maintain their integrity and structure in the absence of a separate support. Furthermore, the films of the present invention may be selected of materials that are edible or ingestible.

### Casting or Depositing the Film Composition

The invention uses processes for making self-supporting films having a substantially uniform distribution of components. The self supporting film is particularly useful for delivery of actives as discussed herein. The processes for making the film are designed to maintain the compositional uniformity of components distributed throughout the film, which is particularly necessary when actives, such as pharmaceutical actives, are incorporated into the film. In the pharmaceutical context, it is essential that the film is compositionally uniform so that it can be divided into individual film dosage units, each dosage unit having the appropriate amount of active when administered, such that regulatory approval can be secured.

The process may further include the preliminary steps of forming a masterbatch premix of an edible water-soluble polymer and water; optionally deaerating the premix (such as by mixing); feeding a predetermining amount of the premix to at least one mixer; adding the nanoparticles to the mixer; and mixing the components to achieve a uniform distribution thereof. Thereafter, the wet film is formed and dried.

Coating or casting methods are particularly useful for the purpose of forming the films of the present invention. Specific examples include reverse roll coating, gravure coating, immersion or dip coating, metering rod or meyer bar coating, slot die or extrusion coating, gap or knife over roll coating, air knife coating, curtain coating, or combinations thereof, especially when a multi-layered film is desired.

Roll coating, or more specifically reverse roll coating, is particularly desired when forming films in accordance with the present invention. This procedure provides excellent control and uniformity of the resulting films, which is desired in the present invention. In this procedure, the coating material is measured onto the applicator roller by the precision setting of the gap between the upper metering roller and the application roller below it. The coating is transferred from the application roller to the substrate as it passes around the support roller adjacent to the application roller. Both three roll and four roll processes are common.

The gravure coating process relies on an engraved roller running in a coating bath, which fills the engraved dots or lines of the roller with the coating material. The excess coating on the roller is wiped off by a doctor blade and the coating is then deposited onto the substrate as it passes between the engraved roller and a pressure roller.

Offset Gravure is common, where the coating is deposited on an intermediate roller before transfer to the substrate.

In the simple process of immersion or dip coating, the substrate is dipped into a bath of the coating, which is normally of a low viscosity to enable the coating to run back into the bath as the substrate emerges.

In the metering rod coating process, an excess of the coating is deposited onto the substrate as it passes over the bath roller. The wire-wound metering rod, sometimes known as a Meyer Bar, allows the desired quantity of the coating to remain on the substrate. The quantity is determined by the diameter of the wire used on the rod.

In the slot die process, the coating is squeezed out by gravity or under pressure through a slot and onto the substrate. If the coating is 100% solids, the process is termed “Extrusion” and in this case, the line speed is frequently much faster than the speed of the extrusion. This enables coatings to be considerably thinner than the width of the slot.

The gap or knife over roll process relies on a coating being applied to the substrate which then passes through a “gap” between a “knife” and a support roller. As the coating and substrate pass through, the excess is scraped off.

Air knife coating is where the coating is applied to the substrate and the excess is “blown off” by a powerful jet from the air knife. This procedure is useful for aqueous coatings.

In the curtain coating process, a bath with a slot in the base allows a continuous curtain of the coating to fall into the gap between two conveyors. The object to be coated is passed along the conveyor at a controlled speed and so receives the coating on its upper face.

### Drying the Film

The drying step can also be a contributing factor with regard to maintaining the uniformity of the film composition. A controlled drying process is particularly important when, in the absence of a viscosity increasing composition or a composition in which the viscosity is controlled, for example by the selection of the polymer, the components within the film may have an increased tendency to aggregate or conglomerate. An alternative method of forming a film with an accurate dosage, that would not necessitate the controlled drying process, would be to cast the films on a predetermined well. With this method, although the components may aggregate, this will not result in the migration of the active to an adjacent dosage form, since each well may define the dosage unit per se.

One process used to make the films is described in U.S. Pat. No. 7,425,292, which is incorporated in its entirety herein by reference. In this process, the films are prepared by rapidly forming a visco-elastic film by applying hot air currents to the film to prevent flow migration and intermolecular forces from creating aggregates or conglomerates thereby maintaining compositional uniform distribution of components in the film; and further drying the visco-elastic film to form a self-supporting film.

The wet film forming matrix first may be fed onto the top side of a surface prior to the application of hot air currents. The wet film is desirably formed from a deaerated matrix within a time period before the active contained therein degrades. The process may further include a step of dividing the dried film into individual dosage units of equal dimensions and compositional make-up. There may be hot air currents applied to the top surface, if desired. In such embodiments, it may be desired that hot air currents be applied to the bottom surface of the film at a higher velocity than to the top surface of the film during drying. Hot air currents applied to dry the top of the films are preferably less than that which would cause surface rippling or skinning. This permits the film to sufficiently thicken in viscosity to lock-in volumetric uniformity while permitting evaporation of water through the non-skinned surface.

When a controlled or rapid drying process is used, liquid carriers are removed from the film in a manner such that the uniformity, or more specifically, the non-self-aggregating uniform heterogeneity, that is obtained in the wet film is maintained.

Desirably, the film is rapidly dried, such that a solid, visco-elastic structure is initially formed and the contents of the film are “locked in”. This can take place within the first few minutes, e.g. about the first 0.5 to about 15 minutes, desirably about the first 10 minutes, and most desirably about the first 4.0 minutes of the drying process. This rapid drying may be achieved by increasing the viscosity of the film at the initiation of the drying process, such as by initially exposing the film to a drying source, such as heat or radiation energy. Rapid drying means that the film product's viscosity begins to develop at the initiation of the drying process to lock in the uniformity of the active content as described above. The rapid increase in viscosity is achieved at the initial stage of drying because the initial rate of heat transfer in the film should be sufficiently high in order to achieve the visco-elastic film formation.

It may be desired to limit the amount of top air flow during this initial drying stage. Controlling the drying in this manner prevents the destruction and reformation of the film's top surface, which results from conventional drying methods. This is accomplished by forming the film and placing it on the top side of a surface having top and bottom sides. Then, heat is initially applied to the bottom side of the film to provide the necessary energy to evaporate or otherwise remove the liquid carrier. The films dried in this manner dry more quickly and evenly as compared to air-dried films, or those dried by conventional drying means. In contrast to an air-dried film that dries first at the top and edges, the films dried by applying heat to the bottom dry simultaneously at the center as well as at the edges. This also prevents settling of ingredients that occurs with films dried by conventional means.

The internal temperature of the film forming matrix during drying is desirably about 100° C. or less, desirably about 70° C. or less, and most desirably about 60° C. or less. It may be desired to dry the film such that the temperature within the film is less than the boiling point of any solvent or solvents that are within the film forming matrix. Further, it is desirable that the temperature within the film forming matrix is maintained below a temperature at which substantial degradation of actives contained within the film will occur. It is noted, however, that the temperature outside of the film may be above the temperature within the film, and in some instances may be substantially higher than the temperature within the film. The interior of the film remains at a temperature below which substantial degradation of the active contained therein occurs. It is generally understood that some degradation of the active may occur, but such degradation should not be of a substantial amount such that the uniformity of the non-degraded active content is outside the uniformity levels set forth above. That is, unit doses cut from the film should not vary from each other or from the target level of active by about 10% of viable, non-degraded active content.

Another method of controlling the drying process, which may be used alone or in combination with other controlled methods as disclosed above includes controlling and modifying the humidity within the drying apparatus where the film is being dried. In this manner, the premature drying of the top surface of the film may be avoided.

Another method of drying tracks that previously set forth by Magoon, which is based on an interesting property of water. Although water transmits energy by conduction and convection both within and to its surroundings, water only radiates energy within and to water. Therefore, the apparatus of Magoon includes a surface onto which the fruit pulp is placed that is transparent to infrared radiation. The underside of the surface is in contact with a temperature controlled water bath. The water bath temperature is desirably controlled at a temperature slightly below the boiling temperature of water. When the wet fruit pulp is placed on the surface of the apparatus, this creates a “refractance window.” This means that infrared energy is permitted to radiate through the surface only to the area on the surface occupied by the fruit pulp, and only until the fruit pulp is dry. The apparatus of Magoon provides the films of the present invention with an efficient drying time reducing the instance of aggregation of the components of the film.

The objective of the drying processes described herein is to provide a method of drying the films that avoids complications, such as the noted “rippling” effect, that are associated with conventional drying methods and which initially dry the upper surface of the film, trapping moisture inside. In conventional oven drying methods, as the moisture trapped inside subsequently evaporates, the top surface is altered by being ripped open and then reformed.

These complications are avoided by the present drying methods, and a uniform film is provided by drying the bottom surface of the film first or otherwise preventing the formation of polymer film formation (skin) on the top surface of the film prior to drying the depth of the film. This may be achieved by applying heat as described above, or alternatively by the introduction of radiation (such as controlled microwaves) to evaporate the water or other polar solvent within the film. In some embodiments, the film is rapidly dried so as to form a visco-elastic structure within the first ten minutes of drying, and more particularly within the first four minutes of drying. Desirably, the film is dried at such a rapid rate that any components, including the nanoparticles, do not undesirably aggregate together. By rapidly drying the wet matrix, a substantial number of the nanoparticles do not have time to agglomerate.

Yet alternatively, drying may be achieved by using balanced fluid flow, such as balanced air flow, where the bottom and top air flows are controlled to provide a uniform film. In such a case, the air flow directed at the top of the film should not create a condition which would cause movement of particles present in the wet film, due to forces generated by the air currents, that is, any top air flow that is present during this drying stage should be insufficient to overcome the inherent viscosity of the film surface. Additionally, any air currents directed at the bottom of the film should desirably be controlled such that the film does not lift up due to forces from the air. There may be more top air currents than bottom air currents, so long as the air currents are controlled so as to avoid skinning, rippling, or movement of particles within the matrix that results in undesirable agglomeration or non-uniformity. Uncontrolled air currents, either above or below the film, can create non-uniformity in the final film products. The humidity level of the area surrounding the top surface may also be appropriately adjusted to prevent premature closure or skinning of the polymer surface.

The present invention yields exceptionally uniform film products when attention is paid to reducing the aggregation of the compositional components. By avoiding the introduction of and eliminating excessive air in the mixing process, selecting polymers and solvents to provide a controllable viscosity and by drying the film in a rapid manner from the bottom up, such films result. Various drying methods include those set forth in U.S. Pat. Nos. 7,425,292 and 7,357,891, which are herein incorporated by reference in their entireties.

The films may have a thickness of from about 0.1 to about 10 mils, or from about 10 to about 30 mils, or greater than about 30 mils.

### Extruding the Film Composition

In alternative embodiments, the film products of the present invention may be formed by extrusion rather than casting or deposition methods. Extrusion is particularly useful for film compositions containing polyethylene oxide-based polymer components, as discussed below. For instance, a single screw extrusion process may be employed in accordance with the present invention. According to such an extrusion process, pressure builds in the polymer melt so that it may be extruded through a die or injected into a mold.

It may be particularly desirable to employ extrusion methods for forming film compositions containing PEO polymer components. These compositions contain PEO or PEO blends in the polymer component, and may be essentially free of added plasticizers, and/or surfactants, and polyalcohols.

The compositions may be extruded as a sheet at processing temperatures of less than about 90° C. Extrusion may proceed by squeezing the film composition through rollers or a die to obtain a uniform matrix. The extruded film composition then is cooled by any mechanism known to those of ordinary skill in the art. For example, chill rollers, air cooling beds, or water cooling beds may be employed. The cooling step is particularly desirable for film compositions containing PEO polymer components because PEO tends to hold heat. The thus formed sheets can be formed into various shapes, as desired.

### Uses of Films

The films of the present invention are well suited for many uses. The high degree of uniformity of the components of the film makes them particularly well suited for incorporating pharmaceuticals. Furthermore, the polymers used in construction of the films may be chosen to allow for a range of disintegration times for the films. A variation or extension in the time over which a film will disintegrate may achieve control over the rate that the active is released, which may allow for a sustained release delivery system. In addition, the films may be used for the administration of nanoparticles to skin and other body surfaces, including those with mucous membranes.

The films may be used to administer nanoparticles through topical, oral, or any other administration desired. The films may also be reconstituted in a suitable liquid carrier and subsequently administered by injection or infusion. Administration may be accomplished by preparing the film as described above, introducing the film to a skin or mucosal surface of a mammal, and wetting the film if necessary, for example. If desired, this film may be prepared and adhered to a second or support layer from which it is removed prior to use, i.e. application to the skin. An adhesive may be used to attach the film to the support or backing material, which may be any of those known in the art, and is preferably not water soluble. If an adhesive is used, it will desirably be an adhesive that does not alter the properties of the active. Mucoadhesive compositions are also useful. The film compositions in many cases serve as mucoadhesives themselves.

The films of the present invention take advantage of the films' tendency to dissolve quickly when wetted, i.e., through contact with a wetting agent such as water or saliva. The nanoparticles may be introduced to a liquid by preparing a film in accordance with the present invention, introducing it to a liquid, and allowing the film to dissolve. This may be used to prepare a liquid dosage form of the nanoparticles, which may then be administered to the user.

### EXAMPLES

The following experiments are examples of combining antiemetic and antimigraine medications into one dosage format which will allow the user to take one dosage unit, and achieve the desired fast onset of pain relief with relief of nausea and/or vomiting associated with a migraine or other central nervous system related pain disorder. This is useful not only to a user, who only needs to take one dosage, but also since only one prescription is needed, which will lead to lower costs and less chance for error in taking the incorrect medicine or out-of-sequence medication.

Examples 1-4 demonstrate the use of multiple films with combinations of them into one film dosage format. Although single-layer films or multi-layer films may be used, in this embodiment, the multi-layer films are used to maintain the first and second active components physically separate from each other. For example, such physical separation may be required when one active component is acidic and the other active component is basic. Any desired method for preparing multi-layered films may be used, including, for example, melt lamination, solvent lamination, adhesion lamination, slot die coating of an already-formed active containing layer, and the like.

Example 5 demonstrates a combination of both an anti-emetic compound and an anti-migraine compound into a single-layer dose. This embodiment is quite useful when the antiemetic compound and the anti-migraine compound are chemically compatible, and thus have no need for physical separation.

Example 6 demonstrates the combination of both the anti-emetic compound and the antimigraine compound into a single dose formulation, with a controlled release coating on one of the actives. This not only allows for a controlled release of that active, but also is useful in maintaining physical separation of the two active components. For example, such physical separation may be required when one active is acidic and the other is basic.

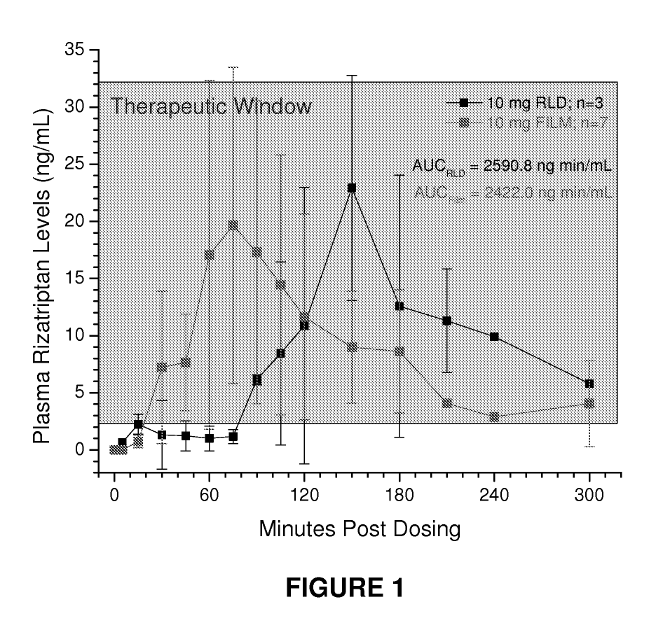

Finally, Examples 7-8 (with accompanying FIGS. 1 & 2) demonstrate an animal study and a clinical human study that have been performed. These studies clearly demonstrate the faster onset of action for the tested anti-migraine agent. As explained in these Examples and seen in the Figures, significant and beneficial results are achieved through administration of the present invention.

### Example 1

### 10 Mg Rizatriptan Active Film Formulation for Lamination into a Combination Product

A film formulation was prepared with the following components:
1. 2.044 g (25.545%) Polyethylene Oxide
2. 1.022 g (12.772%) HPMC
3. 0.510 g (6.375%) Glycerin
4. 0.680 g of Maltitol Syrup containing 0.510 g (6.375%) solids and 0.170 g water
5. 0.040 g (0.500%) Peceol
6. 3.875 g (48.433%) Rizatriptan Benzoate
7. 11.830 g Distilled Water

Components 3, 4, 5 and 7 were added to a fabricated glass bowl. Then a blend of components 1, 2, and 6 was added to the bowl. The contents of the bowl were stirred with a spatula by hand for a short while to obtain mixing. A top equipped with a gate impeller stirrer was placed on the bowl. The solution was prepared as described below using the Degussa Dental Multivac Compact:
I. 40 Minutes, stifling at 175 rpm in a vacuum of 60% (18.5 in Hg);
II. 40 Minutes, stifling at 175 rpm in a vacuum of 90% (26 in Hg);
III. 12 Minutes, stifling at 175 rpm in a vacuum of 95% (27 in Hg);
IV. 8 Minutes, stifling at 175 rpm in a vacuum of 98% (28 in Hg);
V. Distilled water was added to compensate for water lost during the run;
VI. 8 Minutes, stifling at 150 rpm in a vacuum of 100% (29 in Hg).

The solution was cast into wet film using the K-Control Coater with the micrometer adjustable wedge bar set a 455 microns onto the non glossy side of 6330L paper substrate. The film was dried for 24 minutes in an 80 C. convection air oven. The film was cut into 10×22 mm strips which weighed in the 28 to 32 mg range. The film had a moisture content of 1.96%. The dry strip target weight of the strips was 30 mg to yield a dosage of 14.53 mg rizatriptan benzoate which is equivalent to 10 mg of rizatriptan base. The target strip weight to account for moisture content was 30.6 mg.

### Example 2

### 8 Mg Ondansetron Occlusive Film Formulation for Lamination into a Combination Product

A film formulation was prepared with the following components:
1. 6.840 g (28.740%) Polyethylene Oxide
2. 3.420 g (14.370%) HPMC
3. 1.709 g (7.185%) Glycerin
4. 2.279 g Maltitol Syrup containing 1.709 g (7.185%) solids and 0.570 g water
5. 3.173 g (13.330%) Ondansetron Base
6. 2.380 g (10.000%) Peppermint 2303 Flavor
7. 1.916 g (8.050%) Cal Essence 450 PCC
8. 1.666 g (7.000%) Acesulfame K
9. 0.238 g 1.000%) Sodium Bicarbonate
10. 0.238 g (1.000%) Cab-O-Sil M-5P
11. 0.238 g (1.000%) Titanium Dioxide
12. 0.119 g (0.500%) Magna Sweet 100
13. 0.025 g (0.100%) Butylated Hydroxytoluene
14. 0.119 g (0.500%) Peceol
15. 0.010 g (0.040%) FD & C Blue #1 Granular
16. 45.63 g Distilled Water

Components 3, 4, 11, 14, 15, and 16 were added to a fabricated glass bowl. Then a blend of components 1, 2, 8, and 12 was added to the bowl. The contents of the bowl were stirred with a spatula by hand for a short while to obtain mixing. A top equipped with a gate impeller stirrer was placed on the bowl. The solution was prepared as described below using the Degussa Dental Multivac Compact:
I. 40 Minutes, stifling at 125 rpm in a vacuum of 60% (18.5 in Hg);
II. 40 Minutes, stifling at 125 rpm in a vacuum of 90% (26 in Hg);
III. 20 Minutes, stifling at 125 rpm in a vacuum of 95% (27 in Hg);
IV. 12 Minutes, stifling at 125 rpm in a vacuum of 98% (28 in Hg);
V. A blend of components 5, 7, 9, and 10 prepared by grinding in a Magic Blender was added;
VI. 4 Minutes, stifling at 150 rpm in a vacuum of 98% (28 in Hg);
VII. 4 Minutes, stifling at 125 rpm in a vacuum of 98% (28 in Hg);
VIII. A solution of components 6 and 13 was added;
IX. Distilled water was added to compensate for water lost during the run;
X. 4 Minutes, stifling=150 rpm in a vacuum of 100% (29 in Hg);
XI. 4 Minutes, stifling at 125 rpm in a vacuum of 100% (29 in Hg).

The solution was cast into wet film using the K-Control Coater with the micrometer adjustable wedge bar set at 810 microns onto Mylar substrate. The film was dried for 28 minutes in an 80 C. convection air oven. The film was cut into 14×22 mm strips which weighed in the 58 to 63 mg range. The film had a moisture content of 1.59%. The dry strip target weight was 60 mg to yield a dosage of 8 mg of ondansetron base. The target strip weight to account for moisture content was 60.97 mg.

### Example 3

### Heat Lamination of the 10 Mg Rizatriptan Formulation and the 8 Mg Ondansetron Formulation to Yield a Rizatriptan/Ondansetron (10/8 Mg) Combination Formulation

A 10×22 mm film strip of the 10 mg rizatriptan formulation described in Example 1 was placed on a 14×22 mm film strip of the 8 mg ondansetron formulation described in Example 2. These two strips were sandwiched in a folded sheet of HDPE coated 6330L paper substrate and were allowed to pass twice through the GBC Heat Sealer H212 using a heat setting of 4. The laminated, two layer strip was removed from the paper substrate after allowing to cool. The two film strips were successfully laminated into a rizatriptan/ondansetron (10/8 mg) combination product using the heat lamination technique.

### Example 4

### Solvent Lamination of the 10 Mg Rizatriptan Formulation and the 8 Mg Ondansetron Formulation to Yield a Rizatriptan/Ondansetron (10/8 Mg) Combination Formulation

A 10×22 mm film strip of the 10 mg rizatriptan formulation described in Example 1 and a 14×22 mm film strip of the 8 mg ondansetron formulation described in Example 2 were wetted on one surface with a 27% solution of Plasdone K-29/32 (PVP) (ISP Technologies) in ethanol. The two wetted surface of the films were placed together to allow bonding and were placed in an 80 C. convection air oven to dry for 10 minutes. The laminated, two layer strip was removed from the oven after allowing to dry. The two film strips were successfully laminated into a rizatriptan/ondansetron (10/8 mg) combination product using the solvent lamination technique.

### Example 5

### Rizatriptan/Ondansetron (10/8 Mg) Combination Formulation

A film formulation was prepared with the following components:
1. 7.839 g (31.608%) HPMC
2. 4.106 g (16.557%) Polyethylene Oxide
3. 3.981 g Maltitol Syrup containing 2.986 g (12.041%) solids and 0.995 g water
4. 0.992 g (4.000%) Acesulfame K
5. 5.148 g (20.757%) Rizatriptan Benzoate
6. 2.834 g (11.429%) Ondansetron Base
7. 0.850 g (3.429%) Peppermint 2303 Flavor
8. 0.044 g (0.179%) Peceol
9. 54.205 g Distilled Water

Components 3, 8, and 9 were added to a fabricated glass bowl. Then a blend of components 1, 2, 4, and 5 were added to the bowl. The contents of the bowl were stirred with a spatula by hand for short while to obtain mixing. A top equipped with a gate impeller stirrer was placed on the bowl. The bowl was equipped with a Variac controlled heating mantel and the heat was turned on. The solution was prepared as described below using the Degussa Dental Multivac Compact:
I. 8 Minutes, stifling at 125 rpm in a vacuum of 60% (18.5 in Hg) and a temperature of 50 C.
II. 20 Minutes, stifling at 125 rpm in a vacuum of 0% and a temperature of 58 C.
III. The heat was cut off and the heating mantel was removed.
IV. Distilled water was added to compensate for the water lost during the run.
V. 40 Minutes, stifling at 125 rpm in a vacuum of 60% (18.5 in Hg)
VI. 40 Minutes, stifling at 125 rpm in a vacuum of 90% (26 in Hg)
VII. 20 Minutes, stifling at 125 rpm in a vacuum of 95% (27 in Hg)
VIII. Component 6 was added.
IX. 12 Minutes, stifling at 150 rpm in a vacuum of 98% (28 in Hg)
X. Component 7 was added.
XI. Distilled water was added to compensate for water lost during the run.
XII. 8 Minutes, stifling at 150 rpmin a vacuum of 100% (29 in Hg)

The solution was cast into wet film using the K-Control Coater with the micrometer adjustable wedge bar set at 700 microns onto Mylar substrate. The film was dried 24 minutes in an 80 C. convection air oven. The film was cut into 22×20 mm strips which weighed in the 67 to 74 mg range. The film had a moisture content of 0.64%. The dry strip target weight was 70 mg to yield a dosage of 14.53 mg rizatriptan benzoate which is equivalent to 10 mg rizatriptan base and to yield a dosage of 8 mg ondansetron base. The target strip weight to account for moisture content was 70.45 mg.

### Example 6

### Rizatriptan/Ondansetron (10/8 Mg) Combination Formulation with Taste Masking Polymers (NB10014-158)

A film formulation was prepared with the following components:
1. 1.564 g (25.230%) HPMC
2. 0.819 g (13.216%) Polyethylene Oxide
3. 0.795 g Maltitol Syrup containing 0.596 g (9.611%) solids and 0.199 g water
4. 0.248 g (4.000%) Acesulfame K
5. 1.287 g (20.757%) Rizatriptan Benzoate
6. 1.417 g (22.857%) Ondansetron Base With Taste Masking Polymers (50% Ondansetron Base and 50% Taste Masking Polymers) (Particle size less than 60 mesh)
7. 0.213 g (3.429%) Peppermint 2303 Flavor
8. 0.044 g (0.714%) Titanium Dioxide
9. 0.011 g (0.179%) Peceol
10. 0.0004 g (0.007%) FD & C Blue #1 Granular
11. 13.601 g Distilled Water

Components 3, 8, 9, 10, and 11 were added to a fabricated glass bowl. Then a blend of components 1, 2, 4, and 5 was added to the bowl. The contents of the bowl were stirred with a spatula by hand for a short while to obtain mixing. A top equipped with a gate impeller stirrer was placed on the bowl. The bowl was equipped with a Variac controlled heating mantel and the heat was turned on. The solution was prepared as described below using the Degussa Dental Multivac Compact:
I. 8 Minutes, stifling at 125 rpm in a vacuum of 60% (18.5 in Hg) and a temperature of 58 C.
II. 20 Minutes, stifling at 125 rpm in a vacuum of 0% and a temperature of 36 C.
III. The heat was cut off and the heating mantel was removed.
IV. Distilled water was added to compensate for the water lost during the run.
V. 40 Minutes, stifling at 125 rpm in a vacuum of 60% (18.5 in Hg)
VI. 40 Minutes, stifling at 125 rpm in a vacuum of 90% (26 in Hg)
VII. 20 Minutes, stifling at 125 rpm in a vacuum of 95% (27 in Hg)
VIII. 12 Minutes, stifling at 150 rpm in a vacuum of 98% (28 in Hg)
IX. 4 Minutes, stifling at 125 rpmin a vacuum of 100% (29 in Hg)
X. Component 7 was added.
XI. Distilled water was added to compensate for water lost during the run.
XII. 4 Minutes, stifling at 125 rpmin a vacuum of 100% (29 in Hg0
XIII. Component 6 was added.
XIV. Distilled water was added to compensate for water lost during the run.
XV. 8 Minutes, stifling at 150 rpmin a vacuum of 100% (29 in Hg)

The solution was cast into wet film using the K-Control Coater with the micrometer adjustable wedge bar set at 700 microns onto Mylar substrate. The film was dried 26 minutes in an 80 C. convection air oven. The film was cut into 22×20 mm strips which weighed in the 64 to 73 mg range. The film had a moisture content of 1.60%. The dry strip target weight was 70 mg to yield a dosage of 14.53 mg rizatriptan benzoate which is equivalent to 10 mg rizatriptan base and to yield a dosage of 8 mg ondansetron base. The target strip weight to account for moisture content was 71.14 mg.

### Example 7

### Composition and Procedure for a 10 Mg Rizatriptan Formulation and Results from Mini Pig Study

A film composition was prepared according to the formula set forth in Table 1 below:

TABLE 1
10 mg Rizatriptan Base (Added as Benzoate Salt) Formulation

| | 10 mg Rizatriptan Base Formulation | |
|---|---|---|
| | in HPMC/PEO/Maltitol | |
| HPMC | 24.29% | (9.716 mg) |
| PEO | 12.14% | (4.856 mg) |
| Maltitol | 12.14% | (4.856 mg) |
| Sucralose | 7.00% | (2.800 mg) |
| Rizatriptan Benzoate | 36.33% | (14.532 mg)* |
| Peppermint 2303 Flavor | 6.00% | (2.400 mg) |
| Butylated Hydroxytoluene | 0.10% | (0.040 mg) |
| Titanium Dioxide | 1.00% | (0.400 mg) |
| Menthol FP 4594 Flavor | 1.00% | (0.400 mg) |
| % Solids | 30.0 | |
| % Moisture | 2.33 | |
| Dry Target Strip Wt. | 40 | |
| Target Strip Wt. Accounting | 40.954 | mg |
| for % Moisture | | |
| Strip Wt. Range | 39 to 42 | mg |

| | |
|---|---|
| Number of Strips Packaged | 8 |
| Individually in Foil Pouches | |
| Strip Size | 26 mm × 22 mm |

*14.53 mg of Rizatriptan Benzoate is equivalent to 10 mg Rizatriptan Base

The film product was produced as follows: The maltitol syrup, menthol flavor, titanium dioxide, and distilled water were added to a fabricated glass bowl. Then a blend of HPMC E15, polyethylene oxide WSR N80 LEO, sucralose, and rizatriptan benzoate were added to the bowl. The contents of the bowl were stirred with a spatula by hand for a short while to obtain mixing. A top equipped with a gate impeller stirrer was placed on the bowl. The solution was prepared as described below using the Degussa Dental Multivac Compact:
I. 40 Minutes, stifling at 175 rpm in a vacuum of 60% (18.5 in Hg)
II. 40 Minutes, stifling at 175 rpm in a vacuum of 90% (26 in Hg)
III. 12 Minutes, stifling at 125 rpm in a vacuum of 95% (27 in Hg) IV. 8 Minutes, stifling at 125 rpmin a vacuum of 98% (28 in Hg)
V. A solution of butylated hydroxytoluene in peppermint 2303 flavor was added.
VI. Distilled water was added to compensate for water lost during the run.
VII. 8 Minutes, stifling at 125 rpmin a vacuum of 100% (29 in Hg)

The solution was cast into wet film using the K-control Coater with the micrometer adjustable wedge bar set at 320 microns onto the non glossy side of 6330L paper substrate. The film was dried 17 minutes in an 80 C. convection air oven. The film was cut into 26×22 mm strips which weighed in the 39 to 42 mg range. The film had a moisture content of 2.33%. The dry strip target weight was 40 mg to yield a dosage of 14.53 mg of rizatriptan benzoate which is equivalent to 10 mg of rizatriptan base. The target strip weight to account for moisture content was 40.95 mg.

The film was administered to mini pigs, and the plasma rizatriptan levels (ng/mL) was measured every 10 minutes post-administration for a period of 300 minutes. Maxalt was also administered and the plasma rizatriptan levels were measured every 10 minutes post-administration for a period of 300 minutes. The results of the plasma rizatriptan levels are shown in FIG. 1.

As can be seen in FIG. 1, the onset of action into the therapeutic widow occurred at around 15 minutes for the inventive combinatorial product as compared to 70-75 minutes for the RLD (Maxalt). This demonstrates a five-fold quicker onset of activity for the inventive product.

### Example 8

### Composition and Procedure for a 10 Mg Rizatriptan Clinical Study

A film product was prepared using the components set forth in Table 2 below. The formulation was prepared using a procedure similar to that described in Example 5. The resulting film was provided to human subjects by placing the product sublingually in the subject's mouth.

TABLE 2
Film Formulation for Clinical Study

| | | Strength (label claim) | |
|---|---|---|---|
| Component and Quality | | 10 mg | |
| Standard (and Grade, | | Quantity per | |
| if applicable) | Function | unit ( mg) | % |
| Rizatriptan Benzoate | Active | 14.53 | 36.33% |
| Methocel | Film Former | 9.72 | 24.29% |
| Sentry Polyox | Film Former | 4.86 | 12.14% |
| Maltitol Syrup Lycasin | Filler | 4.86 | 12.14% |
| 80/55, NF | | | |
| Acesulfame-k, NF | Sweetener | 2.80 | 7.00% |
| Peppermint #2303 | Flavor | 2.40 | 6.00% |
| Menthol FP4594 | Defoamer | 0.40 | 1.00% |
| Titanium Dioxide, USP | Opacifier | 0.40 | 1.00% |
| Butylated | Antioxidant | 0.04 | 0.10% |
| Hydroxytoluene, NF | | | |
| Total | | 40.0 | 100.0 |

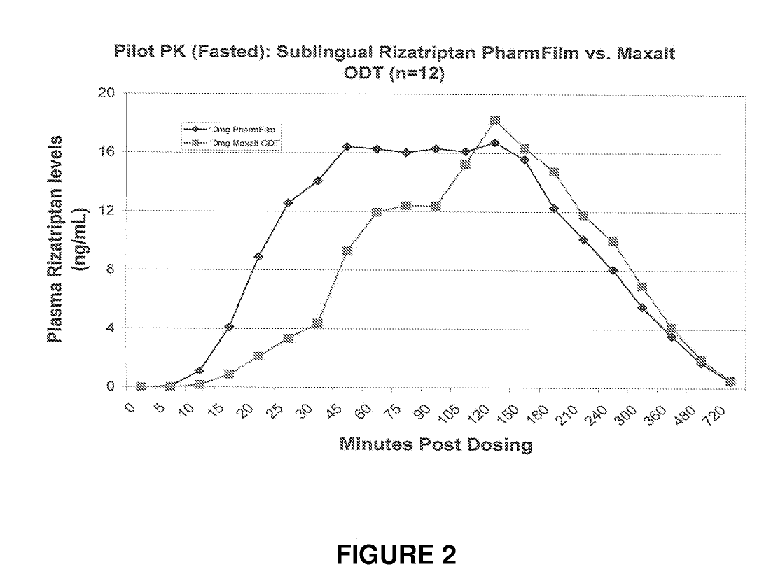

The inventive film formulation was provided to 12 subjects and placed sublingually. The plasma rizatriptan level in the subjects was measured at various intervals over 720 minutes post-administration. Maxalt was provided to 12 subjects. The plasma rizatriptan level in the subjects was measured at various intervals (but the same intervals as for the rizatriptan) over 720 minutes post-administration. The results are shown in FIG. 2.

Much like the mini-pig study of Example 7, the human clinical study shown in FIG. 2 indicates a faster onset of action from the inventive product as compared to a commercial product—Maxalt.

### Example 8

### Composition and Procedure for a 10/8 Mg Rizatriptan/Ondansetron Formulation and Results from Mini Pig Study

A rizatriptan/ondansetron (10/8 mg) combination formulation was prepared without taste masking polymers according to the formula set forth in Table 3 below:

TABLE 3
Rizatriptan/Ondansetron (10/8 mg) Formulation

| Ingredient/Parameter | Amount | | Function |
|---|---|---|---|
| HPMC | 31.608% | (22.126 mg) | Film Former |
| PEO | 16.557% | (11.590 mg) | Film Former |
| Maltitol | 12.041% | (8.429 mg) | Sweetener and |
| | | | Plasticizer |
| Acesulfame K | 4.000% | (2.800 mg) | Sweetener |
| Rizatriptan Benzoate | 20.757% | (14.530 mg) | Active |
| Ondansetron Base | 11.429% | (8.000 mg) | Active |
| Peppermint 2303 Flavor | 3.429% | (2.400 mg) | Flavor |
| Peceol | 0.179% | (0.125 mg) | Defoamer and |
| | | | Deairation |
| % Solids | 31 | | |
| % Moisture | 0.64 | | |
| Dry Strip Target Weight | 70 | mg | |
| Target Strip Weight to | 70.451 | mg | |
| Account for | | | |
| Moisture Content | | | |
| Strip Size | 20 × 22 | mm | |
| Strip Weight Range of Strips | 70 to 71 | mg | |
| Strip Weight Range of Strips | 67 to 74 | mg | |
| Sent Portage | | | |

The film was administered to mini pigs, and the plasma rizatriptan and ondensetron levels (ng/mL) were measured every 30 minutes post-administration for a period of 8 hours.

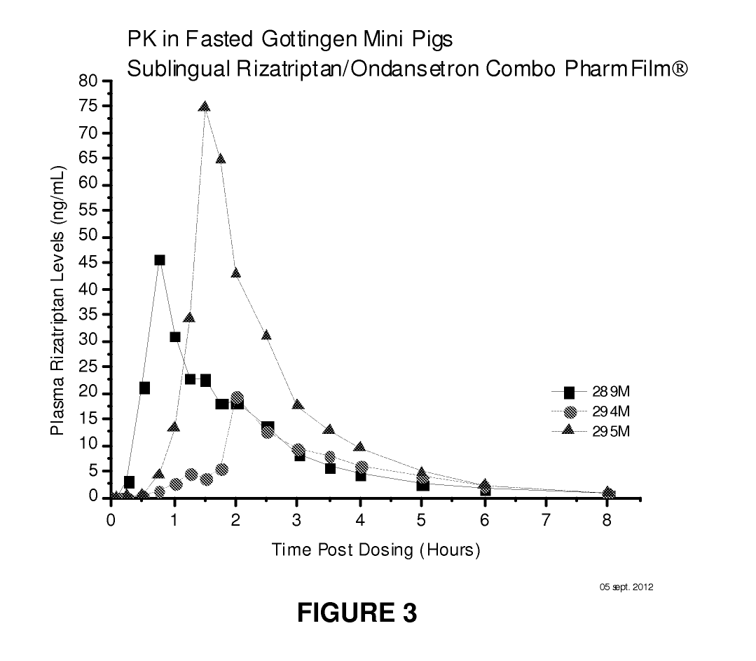

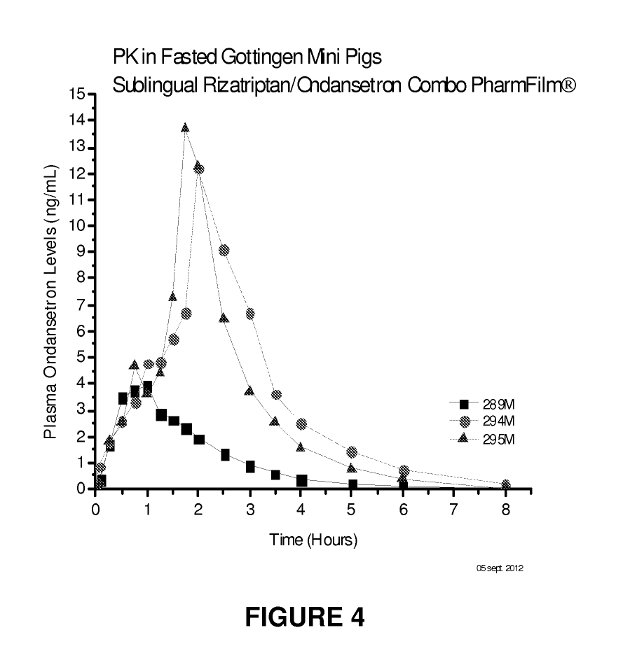

The results of the plasma rizatriptan levels are in FIG. 3 and the results of the plasma ondansetron levels are shown in FIG. 4.

While the Cₘₐₓ levels for the minipig for both the rizatriptan and ondansetron vary from pig to pig, the results clearly show that the active rizatriptan and ondansetron are both readily absorbed when combined together in a combo product. In fact, it appears that rizatriptan is even better absorbed in the combination as two of the pigs showed a much improved absorption at Cₘₐₓ vs. Maxalt from FIG. 1 at Cₘₐₓ.

What is claimed is:

1. A film product comprising:
(a) a polymer component;
(b) a therapeutically effective amount of rizatriptan or a pharmaceutically acceptable salt thereof; and
(c) a therapeutically effective amount of ondansetron or a pharmaceutically acceptable salt thereof.

2. The film product according to claim 1, wherein the rizatriptan or a pharmaceutically acceptable salt thereof is rizatriptan benzoate.

3. The film product according to claim 1, wherein the ondansetron or a pharmaceutically acceptable salt thereof is ondansetron base.

4. The film product according to claim 1, wherein the rizatriptan or a pharmaceutically acceptable salt thereof is rizatriptan benzoate and the ondansetron or a pharmaceutically acceptable salt thereof is ondansetron base.

5. The film product according to claim 4, wherein the weight ratio of rizatriptan benzoate to ondansetron base is about 14.5 to 8.

6. The film product according to claim 1, wherein the polymer component comprises polyethylene oxide and hydroxypropyl methyl cellulose.

7. The film product according to claim 1, wherein the amount of rizatriptan or a pharmaceutically acceptable salt thereof and the amount of ondansetron or a pharmaceutically acceptable salt thereof present in the film product results in a weight ratio of rizatriptan base to ondansetron base of about 10 to 8.

8. The film product according to claim 1, wherein the film product is a dosage unit and the amount of rizatriptan or a pharmaceutically acceptable salt thereof is equivalent to about 10 milligrams of rizatriptan base and the amount of ondansetron or a pharmaceutically acceptable salt thereof is equivalent to about 8 milligrams of ondansetron base.

9. The film product according to claim 8, wherein the film product is a dosage unit and contains about 14.5 milligrams of rizatriptan benzoate and about 8 milligrams of ondansetron base.

10. A film product comprising:
(a) a polymer component comprising polyethylene oxide and hydroxypropyl methyl cellulose;
(b) a therapeutically effective amount of rizatriptan benzoate; and
(c) a therapeutically effective amount of ondansetron base
wherein the weight ratio of rizatriptan benzoate to ondansetron base is about 14.5 to 8.

11. A film dosage unit product comprising:
(a) a polymer component comprising polyethylene oxide and hydroxypropyl methyl cellulose;
(b) about 14.5 milligrams of rizatriptan benzoate; and
(c) about 8 milligrams of ondansetron base.
